SEQUENCE LISTING (26 sequences)

SEQ ID NO: 1 - protein, 1273 aa, synthetic construct
  REGION 1..1273: note = Wuhan-1 S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1273 aa, synthetic construct
  REGION 1..1273: note = S protein variant comprising the S-2P mutation
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1277 aa, synthetic construct
  REGION 1..1277: note = S protein variant is S-2P-VTYA
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFKLKHTKKRQIATDIEMNRLGK

SEQ ID NO: 4 - protein, 1277 aa, synthetic construct
  REGION 1..1277: note = S protein variant is SD614G-2P-VTYA
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFKLKHTKKRQIATDIEMNRLGK

SEQ ID NO: 5 - skipped

SEQ ID NO: 6 - protein, 1275 aa, synthetic construct
  REGION 1..1275: note = S protein variant is SDelta-2P-VTYA
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFKLKHTKKRQIATDIEMNRLGK

SEQ ID NO: 7 - protein, 1247 aa, synthetic construct
  REGION 1..1247: note = S protein variant is SD614G(ECD)-GPI
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKLEPNKGSGTTSGTTRLLSGHTCFTLTGLLGTLVTMGLLTGGS

SEQ ID NO: 8 - protein, 1277 aa, synthetic construct
  REGION 1..1277: note = S protein variant is SD614G-VT
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFKLKHTKKRQIYTDIEMNRLGK

SEQ ID NO: 9 - protein, 1277 aa, synthetic construct
  REGION 1..1277: note = S protein variant is SD614G-VTYA
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFKLKHTKKRQIATDIEMNRLGK

SEQ ID NO: 10 - protein, 1273 aa, synthetic construct
  REGION 1..1273: note = S protein variant is SD614G-2P
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1247 aa, synthetic construct
  REGION 1..1247: note = S protein variant is SD614G-2P(ECD)-GPI
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKLEPNKGSGTTSGTTRLLSGHTCFTLTGLLGTLVTMGLLTGGS

SEQ ID NO: 12 - protein, 1295 aa, synthetic construct
  REGION 1..1295: note = Wuhan-1 N protein
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEGTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKLE

SEQ ID NO: 13 - protein, 688 aa, synthetic construct
  REGION 1..688: note = N protein variant Lamp-N-Lamp protein
MAAPGSARRPLLLLLLLLLLGLMHCASAAMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSGTSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFG

SEQ ID NO: 14 - DNA, 4039 bp, synthetic construct
  misc_feature 1..4039: note = S-2P-VTYA corresponds to the nucleotide sequence
  source 1..4039: mol_type = other DNA
  misc_feature 4039: note = polyA tail attached
gataatctcccgggctggcagcagggccccagcggcaccggtaccatgtttgtatttctggtacttttgccactcgtcagttcccaatgtgttaacctgaccactcgtacgcaactccccccagcatataccaactcatttacgcggggagtatattatccggacaaggtatttaggagttctgtactgcacagcacacaggacctttttcttccttttttttccaacgtgacatggtttcatgcgatacacgtaagtggaaccaatggaactaaaaggttcgataatcctgtacttccgtttaacgatggtgtctatttcgcttctacggagaagtcaaatatcattagaggttggatcttcggtacaactcttgacagtaagacgcaaagtctgctcatcgtaaacaacgcgacgaacgtagtgataaaggtctgcgagtttcagttctgtaatgatccgtttcttggcgtctattaccataagaacaataagagttggatggaaagtgagttccgagtatacagcagcgctaacaattgcacatttgaatatgtctctcaacctttcctgatggacctcgaagggaaacaaggtaacttcaaaaatctccgagaatttgtttttaaaaatatagacggatatttcaagatatactcaaagcacacgccgataaatctggtcagggatctccctcagggtttcagtgcactcgaacctttggtagatttgcctatcggaataaatattacacgctttcagacgcttcttgcgttgcaccggtcttaccttacacctggggacagttcatctggttggacggccggggctgccgcttattacgtcggatatctccaaccaagaacgtttctgcttaaatataatgagaacggcacaataactgacgcggtggattgcgctcttgatcctctctcagagaccaaatgcacgctcaaatcctttactgtcgagaaagggatttatcagaccagcaactttcgcgtgcagcctacagaaagtattgtaagattccctaacattactaacctctgcccgttcggcgaggtatttaatgcaacacgatttgcaagtgtctatgcgtggaatagaaaacgcatttcaaattgtgtcgccgattactccgtactctataacagcgcctcatttagcacgtttaaatgttatggtgtctcccccacaaagctcaatgatctctgcttcactaacgtgtatgctgatagcttcgtgatccgaggtgacgaagtgaggcaaattgctccgggtcaaaccgggaagatcgcagattataactataagttgccagacgatttcactggatgtgttattgcgtggaactctaataacctggactcaaaagtgggagggaattacaattacctctacaggctgtttcgcaaaagcaatttgaaacccttcgagagggacatcagtactgagatataccaggcagggtcaaccccctgcaatggagtcgaaggatttaattgctacttcccgcttcagagctatgggttccagccaacgaacggagtgggctaccaaccctatcgggtcgtagttttgagttttgagctgctccacgcccctgcgacggtttgtggacctaagaagagcactaacctcgtgaaaaataagtgtgtcaattttaattttaatggcctgacagggacaggggtccttacagagtccaacaaaaagtttcttccttttcagcagtttgggagggacatagccgatacaaccgacgccgtccgagatccacaaaccttggaaatattggatatcaccccgtgtagtttcggcggagtcagcgttattaccccagggaccaataccagtaaccaggtagccgtattgtatcaagatgttaattgtactgaagtgcctgtggctatccacgcagaccaactgaccccgacatggcgagtatacagtacgggttctaacgtgtttcaaaccagggctggatgtttgatcggtgcagaacacgttaataacagttacgagtgcgatatcccgatcggtgcgggcatttgtgccagttaccaaacacagacaaacagtccccgccgagcaagaagcgttgcgagtcagtctattatagcttacacgatgtcactgggtgctgaaaacagcgtagcctattctaataattccatcgccatacctacaaacttcaccatatctgtgacgactgaaatcctgccagtaagcatgactaagacatcagtggactgtactatgtatatctgtggcgactccacagagtgtagtaatctgttgctgcaatacgggtctttctgtacccagttgaatcgcgcattgacgggcatagccgttgaacaggacaagaacacgcaagaagtattcgcacaagttaaacaaatctataagactccgcctataaaagactttggtggattcaattttagtcaaatacttcctgatccgtccaagccgagtaaacgaagtttcatagaggatttgttgttcaataaggtgacattggctgacgcaggctttatcaaacagtacggtgactgtctcggagacattgctgcgcgagaccttatatgtgcgcaaaagtttaacggacttactgttttgccgcccttgttgacggacgaaatgatagcacaatatacttccgctctgcttgctggaacaattacttctggctggactttcggagcgggtgctgccctccagataccctttgcgatgcaaatggcttacagattcaacggcatcggagtgactcagaacgtcctgtacgaaaaccaaaagcttatcgcaaatcagtttaactctgcaatcggaaaaatccaagacagcttgtctagtacggctagcgcgcttggaaaacttcaggatgttgttaaccagaacgcgcaagcactcaatacgttggttaagcagttgtcatctaatttcggcgcaatctcttcagtacttaacgatattttgtcaaggctcgacccgccggaagccgaagtgcagatcgatagactcataactgggagattgcagtcccttcaaacctatgttacacagcagttgatacgagcggccgagatcagggcgtctgcgaaccttgccgcaacaaagatgtctgagtgcgttttggggcagtccaaacgcgtggatttctgcggtaaagggtatcaccttatgagttttcctcaatcagcgccccacggggtggttttcctccatgtaacgtatgtacccgcacaagaaaagaacttcaccacagctcccgcaatatgccacgacgggaaggcgcacttcccgagggaaggtgttttcgttagtaacggcacgcattggtttgtgacccagagaaatttttacgagccacagatcatcacaacggacaacacattcgtaagtggaaactgtgatgtagtaattggcatcgtcaacaataccgtctacgaccctctccaaccagaactcgattcttttaaggaggaacttgataaatattttaaaaatcatacaagccccgatgtggacctcggagacatcagcggcattaatgcgagcgtggtcaacatccaaaaggagatagatcgcctcaacgaagttgccaagaatttgaatgagagccttatagatcttcaggaactcggcaagtacgagcaatacattaaatggccttggtacatatggctgggatttattgcagggctgatagccatcgtgatggtgacaataatgctctgttgtatgactagttgttgcagctgccttaaagggtgctgttcttgtgggagttgctgtaagtttaaacttaagcacaccaagaaacggcagatagcgacggatattgaaatgaataggcttgggaaataataactcgagtaactaactaacggatccagtgtccagaccattgtcttccaaccccagctggcctccagaacacccactggccagtcctagagctcctgtccctacccactctttgctacaataaatgctgaatgaatccaagatctaaacccttgcggcc

SEQ ID NO: 15 - DNA, 4033 bp, synthetic construct
  misc_feature 1..4033: note = SDelta-2P-VTYA corresponds to the nucleotide sequence
  source 1..4033: mol_type = other DNA
  misc_feature 4033: note = polyA tail attached
gataatctcccgggctggcagcagggccccagcggcaccggtaccatgtttgtatttctggtacttttgccactcgtcagttcccaatgtgttaacctgcgcactcgtacgcaactccccccagcatataccaactcatttacgcggggagtatattatccggacaaggtatttaggagttctgtactgcacagcacacaggacctttttcttccttttttttccaacgtgacatggtttcatgcgatacacgtaagtggaaccaatggaactaaaaggttcgataatcctgtacttccgtttaacgatggtgtctatttcgcttctacggagaagtcaaatatcattagaggttggatcttcggtacaactcttgacagtaagacgcaaagtctgctcatcgtaaacaacgcgacgaacgtagtgataaaggtctgcgagtttcagttctgtaatgatccgtttcttgacgtctattaccataagaacaataagagttggatggaaagtggagtatacagcagcgctaacaattgcacatttgaatatgtctctcaacctttcctgatggacctcgaagggaaacaaggtaacttcaaaaatctccgagaatttgtttttaaaaatatagacggatatttcaagatatactcaaagcacacgccgataaatctggtcagggatctccctcagggtttcagtgcactcgaacctttggtagatttgcctatcggaataaatattacacgctttcagacgcttcttgcgttgcaccggtcttaccttacacctggggacagttcatctggttggacggccggggctgccgcttattacgtcggatatctccaaccaagaacgtttctgcttaaatataatgagaacggcacaataactgacgcggtggattgcgctcttgatcctctctcagagaccaaatgcacgctcaaatcctttactgtcgagaaagggatttatcagaccagcaactttcgcgtgcagcctacagaaagtattgtaagattccctaacattactaacctctgcccgttcggcgaggtatttaatgcaacacgatttgcaagtgtctatgcgtggaatagaaaacgcatttcaaattgtgtcgccgattactccgtactctataacagcgcctcatttagcacgtttaaatgttatggtgtctcccccacaaagctcaatgatctctgcttcactaacgtgtatgctgatagcttcgtgatccgaggtgacgaagtgaggcaaattgctccgggtcaaaccgggaagatcgcagattataactataagttgccagacgatttcactggatgtgttattgcgtggaactctaataacctggactcaaaagtgggagggaattacaattaccgctacaggctgtttcgcaaaagcaatttgaaacccttcgagagggacatcagtactgagatataccaggcagggtcaaaaccctgcaatggagtcgaaggatttaattgctacttcccgcttcagagctatgggttccagccaacgaacggagtgggctaccaaccctatcgggtcgtagttttgagttttgagctgctccacgcccctgcgacggtttgtggacctaagaagagcactaacctcgtgaaaaataagtgtgtcaattttaattttaatggcctgacagggacaggggtccttacagagtccaacaaaaagtttcttccttttcagcagtttgggagggacatagccgatacaaccgacgccgtccgagatccacaaaccttggaaatattggatatcaccccgtgtagtttcggcggagtcagcgttattacccctgggaccaataccagtaaccaggtagccgtattgtatcaaggagttaattgtactgaagtgcctgtggctatccacgcagaccaactgaccccgacatggcgagtttacagtacgggttctaacgtgtttcaaaccagggctggatgtttgatcggtgcagaacacgttaataacagttacgagtgcgatatcccgatcggtgcgggcatttgtgccagttaccaaacacagacaaacagtagacgccgagcaagaagcgttgcgagtcagtctattatagcttacacgatgtcactgggtgctgaaaacagcgtagcctattctaataattccatcgccatacctacaaacttcaccatatctgtgacgactgaaatcctgccagtaagcatgactaagacatcagtggactgtactatgtatatctgtggcgactccacagagtgtagtaatctgttgctgcaatacgggtctttctgtacccagttgaatcgcgcattgacgggcatagccgttgaacaggacaagaacacgcaagaagtattcgcacaagttaaacaaatctataagactccgcctataaaagactttggtggattcaattttagtcaaatacttcctgatccgtccaagccgagtaaacgaagtttcatagaggatttgttgttcaataaggtgacattggctgacgcaggctttatcaaacagtacggtgactgtctcggagacattgctgcgcgagaccttatatgtgcgcaaaagtttaacggacttactgttttgccgcccttgttgacggacgaaatgatagcacaatatacttccgctctgcttgctggaacaattacttctggctggactttcggagcgggtgctgccctccagataccctttgcgatgcaaatggcttacagattcaacggcatcggagtgactcagaacgtcctgtacgaaaaccaaaagcttatcgcaaatcagtttaactctgcaatcggaaaaatccaagacagcttgtctagtacggctagcgcgcttggaaaacttcagaatgttgttaaccagaacgcgcaagcactcaatacgttggttaagcagttgtcatctaatttcggcgcaatctcttcagtacttaacgatattttgtcaaggctcgacccgccggaagccgaagtgcagatcgatagactcataactgggagattgcagtcccttcaaacctatgttacacagcagttgatacgagcggccgagatcagggcgtctgcgaaccttgccgcaacaaagatgtctgagtgcgttttggggcagtccaaacgcgtggatttctgcggtaaagggtatcaccttatgagttttcctcaatcagcgccccacggggtggttttcctccatgtaacgtatgtacccgcacaagaaaagaacttcaccacagctcccgcaatatgccacgacgggaaggcgcacttcccgagggaaggtgttttcgttagtaacggcacgcattggtttgtgacccagagaaatttttacgagccacagatcatcacaacggacaacacattcgtaagtggaaactgtgatgtagtaattggcatcgtcaacaataccgtctacgaccctctccaaccagaactcgattcttttaaggaggaacttgataaatattttaaaaatcatacaagccccgatgtggacctcggagacatcagcggcattaatgcgagcgtggtcaacatccaaaaggagatagatcgcctcaacgaagttgccaagaatttgaatgagagccttatagatcttcaggaactcggcaagtacgagcaatacattaaatggccttggtacatatggctgggatttattgcagggctgatagccatcgtgatggtgacaataatgctctgttgtatgactagttgttgcagctgccttaaagggtgctgttcttgtgggagttgctgtaagtttaaacttaagcacaccaagaaacggcagatagcgacggatattgaaatgaataggcttgggaaataataactcgagtaactaactaacggatccagtgtccagaccattgtcttccaaccccagctggcctccagaacacccactggccagtcctagagctcctgtccctacccactctttgctacaataaatgctgaatgaatccaagatctaaacccttgcggcc

SEQ ID NO: 16 - protein, 37 aa, synthetic construct
  REGION 1..37: note = GPI anchor-conferring peptide
PNKGSGTTSGTTRLLSGHTCFTLTGLLGTLVTMGLLT

SEQ ID NO: 17 - protein, 6 aa, synthetic construct
  REGION 1..6: note = modification in the C-terminus of the S protein variant
YTDIEM

SEQ ID NO: 18 - protein, 21 aa, synthetic construct
  REGION 1..21: note = VSV-G tail peptide sequence
KLKHTKKRQIYTDIEMNRLGK

SEQ ID NO: 19 - protein, 21 aa, synthetic construct
  REGION 1..21: note = tyrosine-to-alanine substituted form of the VT
KLKHTKKRQIATDIEMNRLGK

SEQ ID NO: 20 - protein, 19 aa, synthetic construct
  REGION 1..19: note = C-terminus of the S protein
KFDEDDSEPVLKGVKLHYT

SEQ ID NO: 21 - DNA, 19 bp, synthetic construct
  misc_feature 1..19: note = T7 promoter
  source 1..19: mol_type = other DNA
taatacgactcactataag

SEQ ID NO: 22 - DNA, 33 bp, synthetic construct
  misc_feature 1..33: note = 33 nucleotide-long 5 prime untranslated region from the CYP2E1 gene
  source 1..33: mol_type = other DNA
ctcccgggctggcagcagggccccagcggcacc

SEQ ID NO: 23 - DNA, 67 bp, synthetic construct
  misc_feature 1..67: note = sequence which contains KpnI and AsiSI sites upstream of a stuffer fragment and followed by an MluI site, two stop codons, and XhoI, and BamHI sites
  source 1..67: mol_type = other DNA
ggtaccgcgatcgccgtatcataaaggccggttacggtacgcgttaatagtgactctcgagggatcc

SEQ ID NO: 24 - DNA, 113 bp, synthetic construct
  misc_feature 1..113: note = 3 prime UTR from the ApoA2 gene
  source 1..113: mol_type = other DNA
agtgtccagaccattgtcttccaaccccagctggcctccagaacacccactggccagtcctagagctcctgtccctacccactctttgctacaataaatgctgaatgaatcca

SEQ ID NO: 25 - DNA, 32 bp, synthetic construct
  misc_feature 1..32: note = sequence containing BglII, NotI and MluI sites
  source 1..32: mol_type = other DNA
agatctaaacccttgcggccgcaacgcgcgcg

SEQ ID NO: 26 - DNA, 48 bp, synthetic construct
  misc_feature 1..48: note = T7 terminator sequence
  source 1..48: mol_type = other DNA
ctagcataaccccttggggcctctaaacgggtcttgaggggttttttg

(19) United States
(12) Patent Application Publication
(10) Pub. No.: US 2025/0002540 A1
(43) Pub. Date: Jan. 2, 2025

(54) EXOSOMAL NUCLEIC ACID VACCINE COMPOSITION FOR PROTECTION AGAINST SARS-COV-2 INFECTION AND DISEASE

(71) Applicants: The Johns Hopkins University, Baltimore, MD (US); Capricor, Inc., Beverly Hills, CA (US)

(72) Inventors: Stephen J. Gould, Baltimore, MD (US); Chenxu Guo, Baltimore, MD (US); Shang-Jui Tsai, Baltimore, MD (US); Linda Marban, Beverly Hills, CA (US); Mafalda Cacciottolo, Beverly Hills, CA (US); Justin Nice, Beverly Hills, CA (US); Arjang Salehi, Beverly Hills, CA (US)

(21) Appl. No.: 18/690,177

(22) PCT Filed: Sep. 12, 2022
(86) PCT No.: PCT/US2022/043230

§ 371 Date: Mar. 7, 2024

Related U.S. Application Data
(60) Provisional application No. 63/243,723, filed on Sep. 13, 2021.

(51) Int. Cl. C07K 14/005; A61K 39/00; C12N 15/88

(52) U.S. Cl. CPC C07K 14/005; C12N 15/88; A61K 2039/55555; A61K 2039/575; C07K 2319/912; C12N 2770/20022; C12N 2770/20034

(57) ABSTRACT

The present invention relates to an extracellular vesicle (EV)-based nucleic acid composition or vaccine (EV-NAV), comprising EVs loaded with polynucleotides each encoding, e.g., the SARS-CoV-2 spike protein, and polynucleotides each encoding, e.g., SARS-CoV-2 nucleocapsid protein, wherein said polynucleotides are designed to be simultaneously expressed, and to induce a humoral immune response and/or a cellular immune response, in a subject. The present invention also relates to compositions and methods for the design, preparation, manufacture, formulation, and therapeutic or prophylactic use of said EV-NAVs, e.g., exosomes loaded with mRNAs encoding multiple surface and cytoplasmic antigens derived from, e.g., SARS-CoV-2, to elicit strong humoral and cellular immune responses.

### CROSS-REFERENCE TO RELATED APPLICATIONS

This application claims benefit of priority under 35 U.S.C. § 119(e) of U.S. Provisional Application No. 63/243,723, filed Sep. 13, 2021. The disclosure of the prior application is considered part of an is herein incorporated by reference in the disclosure of this application in its entirety.

### BACKGROUND OF THE INVENTION

As demonstrated by the COVID-19 outbreak, vaccine development can be a costly, time consuming endeavor that is outpaced by fast-spreading infections. There is therefore a great need for the development of new vaccine production platform technologies that can be modularly adapted to any new infectious agent. This need is also apparent for older infectious agents that mutate so rapidly that new vaccines are needed every year, as is the case for flu. Furthermore, we need improved technologies that generate vaccines that more closely mimic the physicochemical state of the intact virus, which is often vesicular in nature. Consequently, there is a pressing need to develop new vaccines as well as new approaches to combatting infectious diseases. Leading SARS-CoV-2 vaccine candidates target a single protein, spike, but it is unclear whether immunity to this one protein can prevent SARS-CoV-2 infection and disease.

### SUMMARY OF THE INVENTION

The present invention provides, in a first aspect, an extracellular vesicle (“EV”)-based nucleic acid composition, e.g., EV-based nucleic acid vaccine (“EV-NAV”), comprising one or more EVs each loaded with one or more polynucleotides each encoding the Wuhan-1 strain SARS-CoV-2 spike protein (“S protein”) or a variant, or a mutant, of the S protein (“S protein variant”), and one or more polynucleotides each encoding the Wuhan-1 strain SARS-CoV-2 nucleocapsid protein (“N protein”) or a variant, or a mutant, of the N protein (“N protein variant”). In some embodiments, said polynucleotides are configured, or designed, to be simultaneously expressed, and to induce a humoral immune response and/or a cellular immune response, in an animal subject. In some embodiments, said one or more polynucleotides are each ribonucleic acid (“RNA”), e.g., messenger RNA (“mRNA”), or deoxyribonucleic acid (“DNA”), e.g., plasmid DNA. In some embodiments, the EV-based nucleic acid composition further comprises a chemical lipofection reagent. In some embodiments, said chemical lipofection reagent is a polycationic lipid. In some embodiments, said chemical lipofection reagent is an mRNA lipofection reagent, or an mRNA transfection reagent, e.g., Lipofectamine® MessengerMAX™, Lipofectamine® 2000, Lipofectamine® 3000. In some embodiments, the EV-based nucleic acid composition further comprises a physiologically acceptable excipient and/or adjuvant. In some embodiments, said EV is derived from, or obtained or harvested from, 293F cells, cardiospheres, cardiosphere-derived cells (CDCs), activated-specialized tissue-effector cells (ASTECs), or mesenchymal stem cells (MSCs). In some embodiments, said polynucleotide contains a codon-optimized open reading frame (“ORF”). In some embodiments, said codon-optimized ORF is codon-optimized for a mammalian cell, and more preferably for a human cell. In some embodiments, said polynucleotide contains an optimized three prime untranslated region (“3′UTR”) and/or an optimized five prime untranslated region (“5′UTR”). In some embodiments, said polynucleotide contains a polyadenylated tail. In some embodiments, said EVs are exosomes or microvesicles.

“S protein” refers to a protein having the amino acid sequence of Wuhan-1 S protein. Wuhan-1 S protein has the amino acid sequence according to SEQ ID NO:1.

(SEQ ID NO: 1)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKF
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDKVEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFDEDD
SEPVLKGVKLHYT

Variants of S protein have amino acid sequences with at least any of 90%, 91%, 92%, 93%, 94%, 95%, 96%, 97%, 98%, and 99% sequence identity with SEQ ID NO:1 (“S protein variant”).

In certain embodiments, S protein variants bind to human ACE2 protein. In other embodiments, S protein variants bind to antibodies that bind to Wuhan-1 spike protein.

S protein variants include, for example, naturally occurring variants, such as Delta variant, Lambda variant, Mu variant, etc.

S protein variants include, for example, variants having one or more modifications in the C-terminus of the S protein or S-protein variant that improve its cell-surface expression, e.g., comprising removal of one or more retrieval signals that serve to localize the S protein to a specific cellular compartment and/or addition of one or more endoplasmic reticulum (“ER”) export signals.

In some embodiments of the first aspect of the present invention, the S protein variant comprises a di-proline substitution of 986KV987-to-986PP987 (“S-2P”). In some embodiments, the S protein variant comprising the S-2P mutation has the amino acid sequence according to SEQ ID NO:2, or has at least 90%, 91%, 92%, 93%, 94%, 95%, 96%, 97% 98%, or 99% sequence identity with SEQ ID NO:2 while retaining the S-2P mutation.

(SEQ ID NO: 2)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLENKVTLADAGFIKQYGDCLGDIAARDLICAQKF
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFDEDD
SEPVLKGVKLHYT

In some embodiments of the first aspect of the present invention, the S protein variant comprises a modification in its C-terminus that improves its cell-surface expression as compared to the S protein. In some embodiments, the S protein variant further comprises the S-29 mutation. In some embodiments, said modification in the C-terminus of the S protein variant that improves its cell-surface expression comprises removal of one or more retrieval signals that serve to localize the S protein to a specific cellular compartment and/or addition of one or more endoplasmic reticulum (“ER”) export signals. In some embodiments, said modification in the C-terminus of the S protein variant that improves its cell-surface expression comprises removal of one or more retrieval signals that serve to localize the S protein to a specific cellular compartment and/or expression of the extracellular domain of S protein as a fusion protein to a C-terminally positioned glycosylphosphatidylinositol (“GPI”) anchor-conferring peptide. In some embodiments, said specific cellular compartment is the ER, Golgi, endosome, or lysosome. In some embodiments, the GPI anchor-conferring peptide comprises the sequence PNKGSGTTSGTTRLLSGHTCFTLTGLLGTLVTMGLLTCOOH (SEQ ID NO:16). In some embodiments, said modification in the C-terminus of the S protein variant that improves its cell-surface expression comprises the sequence Tyr19-Thr20-Asp21-Ile22-Glu23-Met24 (SEQ ID NO:17) of the vesicular stomatitis virus glycoprotein (“VSV-G”) tail peptide, the VSV-G tail peptide sequence KLKHTKKRQIYTDIEMNRLGKCOOH (SEQ ID NO:18) (“VT”), or the tyrosine-to-alanine substituted form of the VT (“VTYA” [KLKHTKKRQIATDIEMNRLGKCOOH] (SEQ ID NO:19)).

In some embodiments of the first aspect of the present invention, the sequence KFDEDDSEPVLKGVKLHYTCOOH (SEQ ID NO:20) in the C-terminus of the S protein is removed. In some embodiments, the S protein variant comprises an amino acid change of D614G (“SD614G”) and/or a cleavage site mutation of 682RRAR685-to-682GSAG685 (“S-CSM”).

In some embodiments of the first aspect of the present invention, the S protein variant is S-2P-VTYA having the amino acid sequence according to SEQ ID NO:3, or having at least 90%, 91%, 92%, 93%, 94%, 95%, 96%, 97% 98%, or 99% sequence identity with SEQ ID NO:3 while retaining the S-2P and VTYA mutations, or while retaining the S-2P, D614G and VTYA mutations.

(SEQ ID NO: 3)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLENKVTLADAGFIKQYGDCLGDIAARDLICAQKF
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFKLKH
TKKRQIATDIEMNRLGK

In some embodiments of the first aspect of the present invention, the S protein variant is SD614G-2P-VTYA having the amino acid sequence according to SEQ ID NO:4, or having at least 90%, 91%, 92%, 93%, 94%, 95%, 96%, 97% 98%, or 99% sequence identity with SEQ ID NO:4 while retaining the SD614G, S-2P and VTYA mutations.

(SEQ ID NO: 4)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQGVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLENKVTLADAGFIKQYGDCLGDIAARDLICAQKF
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFKLKH
TKKRQIATDIEMNRLGK

In some embodiments of the first aspect of the present invention, the S protein variant is the Delta variant spike protein (“SDelta”) having the amino acid sequence according to SEQ ID NO:5, or having at least 90%, 91%, 92%, 93%, 94%, 95%, 96%, 97% 98%, or 99% amino acid sequence identity with SEQ ID NO:5 while retaining the SDelta mutation, or while retaining the D614G and SDelta mutations.

In some embodiments of the first aspect of the present invention, the S protein variant is SDelta-2P-VTYA having the amino acid sequence according to SEQ ID NO:6, or having at least 90%, 91%, 92%, 93%, 94%, 95%, 96%, 97% 98%, or 99% sequence identity with SEQ ID NO:6 while retaining the SDelta, S-2P and VTYA mutations, or while retaining the SDelta, S-2P, D614G and VTYA mutations.

(SEQ ID NO: 6)
MFVFLVLLPLVSSQCVNLRTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLDVYYHKNNKSWMESGVYSSANNCTFEYVSQPFLMDLEGK
QGNFKNLREFVFKNIDGYFKIYSKHTPINLVRDLPQGFSALEPLV
DLPIGINITRFQTLLALHRSYLTPGDSSSGWTAGAAAYYVGYLQP
RTFLLKYNENGTITDAVDCALDPLSETKCTLKSFTVEKGIYQTSN
FRVQPTESIVRFPNITNLCPFGEVFNATRFASVYAWNRKRISNCV
ADYSVLYNSASFSTFKCYGVSPTKLNDLCFTNVYADSFVIRGDEV
RQIAPGQTGKIADYNYKLPDDFTGCVIAWNSNNLDSKVGGNYNYR
YRLFRKSNLKPFERDISTEIYQAGSKPCNGVEGFNCYFPLQSYGF
QPTNGVGYQPYRVVVLSFELLHAPATVCGPKKSTNLVKNKCVNFN
FNGLTGTGVLTESNKKFLPFQQFGRDIADTTDAVRDPQTLEILDI
TPCSFGGVSVITPGTNTSNQVAVLYQGVNCTEVPVAIHADQLTPT
WRVYSTGSNVFQTRAGCLIGAEHVNNSYECDIPIGAGICASYQTQ
TNSRRRARSVASQSIIAYTMSLGAENSVAYSNNSIAIPTNFTISV
TTEILPVSMTKTSVDCTMYICGDSTECSNLLLQYGSFCTQLNRAL
TGIAVEQDKNTQEVFAQVKQIYKTPPIKDFGGFNFSQILPDPSKP
SKRSFIEDLLFNKVTLADAGFIKQYGDCLGDIAARDLICAQKFNG
LTVLPPLLTDEMIAQYTSALLAGTITSGWTFGAGAALQIPFAMQM
AYRENGIGVTQNVLYENQKLIANQFNSAIGKIQDSLSSTASALGK
LQNVVNQNAQALNTLVKQLSSNFGAISSVLNDILSRLDPPEAEVQ
IDRLITGRLQSLQTYVTQQLIRAAEIRASANLAATKMSECVLGQS
KRVDFCGKGYHLMSFPQSAPHGVVFLHVTYVPAQEKNFTTAPAIC
HDGKAHFPREGVFVSNGTHWFVTQRNFYEPQIITTDNTFVSGNCD
VVIGIVNNTVYDPLQPELDSFKEELDKYFKNHTSPDVDLGDISGI
NASVVNIQKEIDRLNEVAKNLNESLIDLQELGKYEQYIKWPWYIW
LGFIAGLIAIVMVTIMLCCMTSCCSCLKGCCSCGSCCKFKLKHTK
KRQIATDIEMNRLGK

In some embodiments of the first aspect of the present invention, the S protein variant is SD614G(ECD)-GPI having the amino acid sequence according to SEQ ID NO:7, or having at least 90%, 91%, 92%, 93%, 94%, 95%, 96%, 97% 98%, or 99% sequence identity with SEQ ID NO:7 while retaining the SD614G, S-2P and GPI anchor mutations.

(SEQ ID NO: 7)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQGVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKF
NGLTVLPPLLTDEMIAQYTSALLAGTITSGWTFGAGAALQIPFAM
QMAYRFNGIGVTQNVLYENQKLIANQFNSAIGKIQDSLSSTASAL
GKLQDVVNQNAQALNTLVKQLSSNFGAISSVLNDILSRLDKVEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKLEPNKGSGTT
SGTTRLLSGHTCFTLTGLLGTLVTMGLLTGGS

In some embodiments of the first aspect of the present invention, the S protein variant is SD614G-VT having the amino acid sequence according to SEQ ID NO:8, or having at least 90%, 91%, 92%, 93%, 94%, 95%, 96%, 97% 98%, or 99% sequence identity with SEQ ID NO:8 while retaining the SD614G and VT mutations.

(SEQ ID NO: 8)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQGVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKF
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDKVEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFKLKH
TKKRQIYTDIEMNRLGK

In some embodiments of the first aspect of the present invention, the S protein variant is SD614G-VTYA having the amino acid sequence according to SEQ ID NO:9, or having at least 90%, 91%, 92%, 93%, 94%, 95%, 96%, 97% 98%, or 99% sequence identity with SEQ ID NO:9 while retaining the SD614G and VTYA mutations.

(SEQ ID NO: 9)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQGVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKF
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDKVEAE
VQIDRLITGRLQSLQTYVTQQLIRAAEIRASANLAATKMSECVLG
OSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFKLKH
TKKRQIATDIEMNRLGK

In some embodiments of the first aspect of the present invention, the S protein variant is SD614G-2P having the amino acid sequence according to SEQ ID NO:10, or having at least 90%, 91%, 92%, 93%, 94%, 95%, 96%, 97% 98%, or 99% sequence identity with SEQ ID NO:10 while retaining the SD614G and S-2P mutations.

(SEQ ID NO: 10)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQGVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKF
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFDEDD
SEPVLKGVKLHYT

In some embodiments of the first aspect of the present invention, the S protein variant is SD614G-2P(ECD)-GPI having the amino acid sequence according to SEQ ID NO:11, or having at least 90%, 91%, 92%, 93%, 94%, 95%, 96%, 97% 98%, or 99% sequence identity with SEQ ID NO:11 while retaining the SD614G, S-2P and GPI anchor mutations.

(SEQ ID NO: 11)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQGVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLENKVTLADAGFIKQYGDCLGDIAARDLICAQKF
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKLEPNKGSGTT
SGTTRLLSGHTCFTLTGLLGTLVTMGLLTGGS

“N protein” refers to a protein having the amino acid sequence of Wuhan-1 N protein. Wuhan-1 N protein has the amino acid sequence according to SEQ ID NO:12.

(SEQ ID NO: 12)
MGVQVETISPGDGRTFPKRGQTCVVHYTGMLEDGKKVDSSRDRNK
PFKFMLGKQEVIRGWEEGVAQMSVGQRAKLTISPDYAYGATGHPG
IIPPHATLVFDVELLKPEGTQLPPAYTNSFTRGVYYPDKVFRSSV
LHSTQDLFLPFFSNVTWFHAIHVSGTNGTKRFDNPVLPFNDGVYF
ASTEKSNIIRGWIFGTTLDSKTQSLLIVNNATNVVIKVCEFQFCN
DPFLGVYYHKNNKSWMESEFRVYSSANNCTFEYVSQPFLMDLEGK
QGNFKNLREFVFKNIDGYFKIYSKHTPINLVRDLPQGFSALEPLV
DLPIGINITRFQTLLALHRSYLTPGDSSSGWTAGAAAYYVGYLQP
RTFLLKYNENGTITDAVDCALDPLSETKCTLKSFTVEKGIYQTSN
FRVQPTESIVRFPNITNLCPFGEVFNATRFASVYAWNRKRISNCV
ADYSVLYNSASFSTFKCYGVSPTKLNDLCFTNVYADSFVIRGDEV
RQIAPGQTGKIADYNYKLPDDFTGCVIAWNSNNLDSKVGGNYNYL
YRLFRKSNLKPFERDISTEIYQAGSTPCNGVEGENCYFPLQSYGF
QPTNGVGYQPYRVVVLSFELLHAPATVCGPKKSTNLVKNKCVNFN
FNGLTGTGVLTESNKKFLPFQQFGRDIADTTDAVRDPQTLEILDI
TPCSFGGVSVITPGTNTSNQVAVLYQDVNCTEVPVAIHADQLTPT
WRVYSTGSNVFQTRAGCLIGAEHVNNSYECDIPIGAGICASYQTQ
TNSPRRARSVASQSIIAYTMSLGAENSVAYSNNSIAIPTNFTISV
TTEILPVSMTKTSVDCTMYICGDSTECSNLLLQYGSFCTQLNRAL
TGIAVEQDKNTQEVFAQVKQIYKTPPIKDFGGFNFSQILPDPSKP
SKRSFIEDLLFNKVTLADAGFIKQYGDCLGDIAARDLICAQKFNG
LTVLPPLLTDEMIAQYTSALLAGTITSGWTFGAGAALQIPFAMQM
AYRFNGIGVTQNVLYENQKLIANQFNSAIGKIQDSLSSTASALGK
LQDVVNQNAQALNTLVKQLSSNFGAISSVLNDILSRLDKVEAEVQ
IDRLITGRLQSLQTYVTQQLIRAAEIRASANLAATKMSECVLGQS
KRVDFCGKGYHLMSFPQSAPHGVVFLHVTYVPAQEKNFTTAPAIC
HDGKAHFPREGVFVSNGTHWFVTQRNFYEPQIITTDNTFVSGNCD
VVIGIVNNTVYDPLQPELDSFKEELDKYFKNHTSPDVDLGDISGI
NASVVNIQKEIDRLNEVAKNLNESLIDLQELGKLE

Variants of N protein have amino acid sequences with at least any of 90%, 91%, 92%, 93%, 94%, 95%, 96%, 97%, 98%, and 99% sequence identity with SEQ ID NO:12 (“N protein variant”).

In certain embodiments, N protein variants bind to antibodies that bind to Wuhan-1 N protein.

In some embodiments of the first aspect of the present invention, the N protein variant is Lamp-N-Lamp protein (“LNL protein”) having the amino acid sequence according to SEQ ID NO:13, or having at least 90%, 91%, 92%, 93%, 94%, 95%, 96%, 97% 98%, or 99% amino acid sequence identity with SEQ ID NO:13 while retaining the sequences derived from the Lamp protein.

(SEQ ID NO: 13)
MAAPGSARRPLLLLLLLLLLGLMHCASAAMFMVKNGNGTACIMAN
FSAAFSVNYDTKSGPKNMTFDLPSDATVVLNRSSCGKENTSDPSL
VIAFGRGHTLTLNFTRNATRYSVQLMSFVYNLSDTHLFPNASSKE
IKTVESITDIRADIDKKYRCVSGTQVHMNNVTVTLHDATIQAYLS
NSSFSRGETRCEQDRPSPTTAPPAPPSPSPSPVPKSPSVDKYNVS
GTNGTCLLASMGLQLNLTYERKDNTTVTRLLNINPNKTSASGSCG
AHLVTLELHSEGTTVLLFQFGMNASSSRFFLOGIQLNTILPDARD
PAFKAANGSLRALQATVGNSYKCNAEEHVRVTKAFSVNIFKVWVQ
AFKVEGGQFGSGTSDNGPQNQRNAPRITFGGPSDSTGSNONGERS
GARSKORRPQGLPNNTASWFTALTQHGKEDLKFPRGQGVPINTNS
SPDDQIGYYRRATRRIRGGDGKMKDLSPRWYFYYLGTGPEAGLPY
GANKDGIIWVATEGALNTPKDHIGTRNPANNAAIVLQLPQGTTLP
KGFYAEGSRGGSQASSRSSSRSRNSSRNSTPGSSRGTSPARMAGN
GGDAALALLLLDRLNQLESKMSGKGQQQQGQTVTKKSAAEASKKP
RQKRTATKAYNVTQAFGRRGPEQTQGNFGDQELIRQGTDYKHWPQ
IAQFAPSASAFFG

In some embodiments of the first aspect of the present invention, the mRNA encoding S-2P-VTYA corresponds to the nucleotide sequence according to SEQ ID NO:14, or has at least 90%, 91%, 92%, 93%, 94%, 95%, 96%, 97% 98%, or 99% amino acid sequence identity with SEQ ID NO:14 while retaining the S-2P and VTYA mutations.

(SEQ ID NO: 14)
gataatctcccgggctggcagcagggccccagcggcaccggtacc
ATGTTTGTATTTCTGGTACTTTTGCCACTCGTCAGTTCCCAATGT
GTTAACCTGACCACTCGtACgCAACTCCCCCCAGCATATACCAAC
TCATTTACGCGGGGAGTATATTATCCGGACAAGGTATTTAGGAGT
TCTGTACTGCACAGCACACAGGACCTTTTTCTTCCTTTTTTTTCC
AACGTGACATGGTTTCATGCGATACACGTAAGTGGAACCAATGGA
ACTAAAAGGTTCGATAATCCTGTACTTCCGTTTAACGATGGTGTC
TATTTCGCTTCTACGGAGAAGTCAAATATCATTAGAGGTTGGATC
TTCGGTACAACTCTTGACAGTAAGACGCAAAGTCTGCTCATCGTA
AACAACGCGACGAACGTAGTGATAAAGGTCTGCGAGTTTCAGTTC
TGTAATGATCCGTTTCTTGGCGTCTATTACCATAAGAACAATAAG
AGTTGGATGGAAAGTGAGTTCCGAGTATACAGCAGCGCTAACAAT
TGcACATTTGAATATGTCTCTCAACCTTTCCTGATGGACCTCGAA
GGGAAACAAGGTAACTTCAAAAATCTCCGAGAATTTGTTTTTAAA
AATATAGACGGATATTTCAAGATATACTCAAAGCACACGCCGATA
AATCTGGTCAGGGATCTCCCTCAGGGTTTCAGTGCACTCGAACCT
TTGGTAGATTTGCCTATCGGAATAAATATTACACGCTTTCAGACG
CTTCTTGCGTTGCACCGGTCTTACCTTACACCTGGGGACAGTTCA
TCTGGTTGGACGGCCGGGGCTGCCGCTTATTACGTCGGATATCTC
CAACCAAGAACGTTTCTGCTTAAATATAATGAGAACGGCACAATA
ACTGACGCGGTGGATTGCGCTCTTGATCCTCTCTCAGAGACCAAA
TGCACGCTCAAATCCTTTACTGTCGAGAAAGGGATTTATCAGACC
AGCAACTTTCGCGTGCAGCCTACAGAAAGTATTGTAAGATTCCCT
AACATTACTAACCTCTGCCCGTTCGGCGAGGTATTTAATGCAACA
CGATTTGCAAGTGTCTATGCGTGGAATAGAAAACGCATTTCAAAT
TGTGTCGCCGATTACTCCGTACTCTATAACAGCGCCTCATTTAGC
ACGTTTAAATGTTATGGTGTCTCCCCCACAAAGCTCAATGATCTC
TGCTTCACTAACGTGTATGCTGATAGCTTCGTGATCCGAGGTGAC
GAAGTGAGGCAAATTGCTCCGGGTCAAACCGGGAAGATCGCAGAT
TATAACTATAAGTTGCCAGACGATTTCACTGGATGTGTTATTGCG
TGGAACTCTAATAACCTGGACTCAAAAGTGGGAGGGAATTACAAT
TACCTCTACAGGCTGTTTCGCAAAAGCAATTTGAAACCCTTCGAG
AGGGACATCAGTACTGAGATATACCAGGCAGGGTCAACCCCCTGC
AATGGAGTCGAAGGATTTAATTGCTACTTCCCGCTTCAGAGCTAT
GGGTTCCAGCCAACGAACGGAGTGGGcTACCAACCCTATCGGGTC
GTAGTTTTGAGTTTTGAGCTGCTCCACGCCCCTGCGACGGTTTGT
GGACCTAAGAAGAGCACTAACCTCGTGAAAAATAAGTGTGTCAAT
TTTAATTTTAATGGCCTGACAGGGACAGGGGTCCTTACAGAGTCC
AACAAAAAGTTTCTTCCTTTTCAGCAGTTTGGGAGGGACATAGCC
GATACAACCGACGCCGTCCGAGATCCACAAACCTTGGAAATATTG
GATATCACCCCGTGTAGTTTCGGCGGAGTCAGCGTTATTACCCCA
GGgACCAATACCAGTAACCAgGTAGCCGTATTGTATCAAGATGTT
AATTGTACTGAAGTGCCTGTGGCTATCCACGCAGACCAACTGACC
CCGACATGGCGAGTATACAGTACGGGTTCTAACGTGTTTCAAACC
AGGGCTGGATGTTTGATCGGTGCAGAACACGTTAATAACAGTTAC
GAGTGCGATATCCCGATCGGTGCGGGCATTTGTGCCAGTTACCAA
ACACAGACAAACAGTCCCCGCCGAGCAAGAAGCGTTGCGAGTCAG
TCTATTATAGCTTACACGATGTCACTGGGTGCTGAAAACAGCGTA
GCCTATTCTAATAATTCCATCGCCATACCTACAAACTTCACCATA
TCTGTGACGACTGAAATCCTGCCAGTAAGCATGACTAAGACATCA
GTgGACTGTACTATGTAtATCTGTGGCGACTCCACAGAGTGTAGT
AATCTGTTGCTGCAATACGGGTCTTTCTGTACCCAGTTGAATCGC
GCATTGACGGGCATAGCCGTTGAACAGGACAAGAACACGCAAGAA
GTATTCGCACAAGTTAAACAAATCTATAAGACTCCGCCTATAAAA
GACTTTGGTGGATTCAATTTTAGTCAAATACTTCCTGATCCGTCC
AAGCCGAGTAAACGAAGTTTCATAGAGGATTTGTTGTTCAATAAG
GTGACATTGGCTGACGCAGGCTTTATCAAACAGTACGGTGACTGT
CTCGGAGACATTGCTGCGCGAGAcCTTATATGTGCGCAAAAGTTT
AACGGACTTACTGTTTTGCCGCCCTTGTTGACGGACGAAATGATA
GCACAATATACTTCCGCTCTGCTTGCTGGAACAATTACTTCTGGC
TGGACTTTCGGAGCGGGTGCTGCCCTCCAGATACCCTTTGCGATG
CAAATGGCTTACAGATTCAACGGCATCGGAGTGACTCAGAACGTC
CTGTACGAAAACCAAAAGCTTATCGCAAATCAGTTTAACTCTGCA
ATCGGAAAAATCCAAGACAGCTTGTCTAGTACGGCTAGCGCGCTT
GGAAAACTTCAGGATGTTGTTAACCAGAACGCGCAAGCACTCAAT
ACGTTGGTTAAGCAGTTGTCATCTAATTTCGGCGCAATCTCTTCA
GTACTTAACGATATTTTGTCAAGGCTCGACccGccGGAAGCCGAA
GTGCAGATCGATAGACTCATAACTGGGAGATTGCAGTCCCTTCAA
ACCTATGTTACACAGCAGTTGATACGAGCGGCCGAGATCAGGGCG
TCTGCGAACCTTGCCGCAACAAAGATGTCTGAGTGCGTTTTGGGG
CAGTCCAAACGCGTGGATTTCTGCGGTAAAGGGTATCACCTTATG
AGTTTTCCTCAATCAGCGCCCCACGGGGTGGTTTTCCTCCATGTA
ACGTATGTACCCGCACAAGAAAAGAACTTCACCACAGCTCCCGCA
ATATGCCACGACGGGAAGGCGCACTTCCCGAGGGAAGGTGTTTTC
GTTAGTAACGGCACGCATTGGTTTGTGACCCAGAGAAATTTTTAC
GAGCCACAGATCATCACAACGGACAACACATTCGTAAGTGGAAAC
TGTGATGTAGTAATTGGCATCGTCAACAATACCGTCTACGACCCT
CTCCAACCAGAACTCGATTCTTTTAAGGAGGAACTTGATAAATAT
TTTAAAAATCATACAAGCCCCGATGTGGACCTCGGAGACATCAGC
GGCATTAATGCGAGCGTGGTCAACATCCAAAAGGAGATAGATCGC
CTCAACGAAGTTGCCAAGAATTTGAATGAGAGCCTTATaGATCTT
CAGGAACTCGGCAAGTACGAGCAATACATTAAATGGCCTTGGTAC
ATATGGCTGGGATTTATTGCAGGGCTGATAGCCATCGTGATGGTG
ACAATAATGCTCTGTTGTATGACTAGTTGTTGCAGCTGCCTTAAA
GGGTGCTGTTCTTGTGGGAGTTGCTGTAAGTTTAAACTTAAGCAC
ACCAAGAAACGGCAGATAgcgACGGATATTGAAATGAATAGGCTT
GGGAAAtaataactcgagtaactaactaacggatccagtgtccag
accattgtcttccaaccccagctggcctccagaacacccactggc
cagtcctagagctcctgtccctacccactctttgctacaataaat
gctgaatgaatccaagatctaaacccttgcggcc.
polyA tail

In some embodiments of the first aspect of the present invention, the mRNA encoding SDelta-2P-VTYA corresponds to the nucleotide sequence according to SEQ ID NO: 15, or has at least 90%, 91%, 92%, 93%, 94%, 95%, 96%, 97% 98%, or 99% sequence identity with SEQ ID NO:15 while retaining the SDelta, S-2P and VTYA mutations.

(SEQ ID NO: 15)
gataatctcccgggctggcagcagggccccagcggcaccggtacc
ATGTTTGTATTTCTGGTACTTTTGCCACTCGTCAGTTCCCAATGT
GTTAACCTGcgCACTCGtACgCAACTCCCCCCAGCATATACCAAC
TCATTTACGCGGGGAGTATATTATCCGGACAAGGTATTTAGGAGT
TCTGTACTGCACAGCACACAGGACCTTTTTCTTCCTTTTTTTTCC
AACGTGACATGGTTTCATGCGATACACGTAAGTGGAACCAATGGA
ACTAAAAGGTTCGATAATCCTGTACTTCCGTTTAACGATGGTGTC
TATTTCGCTTCTACGGAGAAGTCAAATATCATTAGAGGTTGGATC
TTCGGTACAACTCTTGACAGTAAGACGCAAAGTCTGCTCATCGTA
AACAACGCGACGAACGTAGTGATAAAGGTCTGCGAGTTTCAGTTC
TGTAATGATCCGTTTCTTGaCGTCTATTACCATAAGAACAATAAG
AGTTGGATGGAAAGTgGAGTATACAGCAGCGCTAACAATTGcACA
TTTGAATATGTCTCTCAACCTTTCCTGATGGACCTCGAAGGGAAA
CAAGGTAACTTCAAAAATCTCCGAGAATTTGTTTTTAAAAATATA
GACGGATATTTCAAGATATACTCAAAGCACACGCCGATAAATCTG
GTCAGGGATCTCCCTCAGGGTTTCAGTGCACTCGAACCTTTGGTA
GATTTGCCTATCGGAATAAATATTACACGCTTTCAGACGCTTCTT
GCGTTGCACCGGTCTTACCTTACACCTGGGGACAGTTCATCTGGT
TGGACGGCCGGGGCTGCCGCTTATTACGTCGGATATCTCCAACCA
AGAACGTTTCTGCTTAAATATAATGAGAACGGCACAATAACTGAC
GCGGTGGATTGCGCTCTTGATCCTCTCTCAGAGACCAAATGCACG
CTCAAATCCTTTACTGTCGAGAAAGGGATTTATCAGACCAGCAAC
TTTCGCGTGCAGCCTACAGAAAGTATTGTAAGATTCCCTAACATT
ACTAACCTCTGCCCGTTCGGCGAGGTATTTAATGCAACACGATTT
GCAAGTGTCTATGCGTGGAATAGAAAACGCATTTCAAATTGTGTC
GCCGATTACTCCGTACTCTATAACAGCGCCTCATTTAGCACGTTT
AAATGTTATGGTGTCTCCCCCACAAAGCTCAATGATCTCTGCTTC
ACTAACGTGTATGCTGATAGCTTCGTGATCCGAGGTGACGAAGTG
AGGCAAATTGCTCCGGGTCAAACCGGGAAGATCGCAGATTATAAC
TATAAGTTGCCAGACGATTTCACTGGATGTGTTATTGCGTGGAAC
TCTAATAACCTGGACTCAAAAGTGGGAGGGAATTACAATTACCgC
TACAGGCTGTTTCGCAAAAGCAATTTGAAACCCTTCGAGAGGGAC
ATCAGTACTGAGATATACCAGGCAGGGTCAAaaCCCTGCAATGGA
GTCGAAGGATTTAATTGCTACTTCCCGCTTCAGAGCTATGGGTTC
CAGCCAACGAACGGAGTGGGcTACCAACCCTATCGGGTCGTAGTT
TTGAGTTTTGAGCTGCTCCACGCCCCTGCGACGGTTTGTGGACCT
AAGAAGAGCACTAACCTCGTGAAAAATAAGTGTGTCAATTTTAAT
TTTAATGGCCTGACAGGGACAGGGGTCCTTACAGAGTCCAACAAA
AAGTTTCTTCCTTTTCAGCAGTTTGGGAGGGACATAGCCGATACA
ACCGACGCCGTCCGAGATCCACAAACCTTGGAAATATTGGATATC
ACCCCGTGTAGTTTCGGCGGAGTCAGCGTTATTACCCCtGGgACC
AATACCAGTAACCAgGTAGCCGTATTGTATCAAGgaGTTAATTGT
ACTGAAGTGCCTGTGGCTATCCACGCAGACCAACTGACCCCGACA
TGGCGAGTtTACAGTACGGGTTCTAACGTGTTTCAAACCAGGGCT
GGATGTTTGATCGGTGCAGAACACGTTAATAACAGTTACGAGTGC
GATATCCCGATCGGTGCGGGCATTTGTGCCAGTTACCAAACACAG
ACAAACAGTagaCGCCGAGCAAGAAGCGTTGCGAGTCAGTCTATT
ATAGCTTACACGATGTCACTGGGTGCTGAAAACAGCGTAGCCTAT
TCTAATAATTCCATCGCCATACCTACAAACTTCACCATATCTGTG
ACGACTGAAATCCTGCCAGTAAGCATGACTAAGACATCAGTgGAC
TGTACTATGTAtATCTGTGGCGACTCCACAGAGTGTAGTAATCTG
TTGCTGCAATACGGGTCTTTCTGTACCCAGTTGAATCGCGCATTG
ACGGGCATAGCCGTTGAACAGGACAAGAACACGCAAGAAGTATTC
GCACAAGTTAAACAAATCTATAAGACTCCGCCTATAAAAGACTTT
GGTGGATTCAATTTTAGTCAAATACTTCCTGATCCGTCCAAGCCG
AGTAAACGAAGTTTCATAGAGGATTTGTTGTTCAATAAGGTGACA
TTGGCTGACGCAGGCTTTATCAAACAGTACGGTGACTGTCTCGGA
GACATTGCTGCGCGAGAcCTTATATGTGCGCAAAAGTTTAACGGA
CTTACTGTTTTGCCGCCCTTGTTGACGGACGAAATGATAGCACAA
TATACTTCCGCTCTGCTTGCTGGAACAATTACTTCTGGCTGGACT
TTCGGAGCGGGTGCTGCCCTCCAGATACCCTTTGCGATGCAAATG
GCTTACAGATTCAACGGCATCGGAGTGACTCAGAACGTCCTGTAC
GAAAACCAAAAGCTTATCGCAAATCAGTTTAACTCTGCAATCGGA
AAAATCCAAGACAGCTTGTCTAGTACGGCTAGCGCGCTTGGAAAA
CTTCAGaATGTTGTTAACCAGAACGCGCAAGCACTCAATACGTTG
GTTAAGCAGTTGTCATCTAATTTCGGCGCAATCTCTTCAGTACTT
AACGATATTTTGTCAAGGCTCGACccGccGGAAGCCGAAGTGCAG
ATCGATAGACTCATAACTGGGAGATTGCAGTCCCTTCAAACCTAT
GTTACACAGCAGTTGATACGAGCGGCCGAGATCAGGGCGTCTGCG
AACCTTGCCGCAACAAAGATGTCTGAGTGCGTTTTGGGGCAGTCC
AAACGCGTGGATTTCTGCGGTAAAGGGTATCACCTTATGAGTTTT
CCTCAATCAGCGCCCCACGGGGTGGTTTTCCTCCATGTAACGTAT
GTACCCGCACAAGAAAAGAACTTCACCACAGCTCCCGCAATATGC
CACGACGGGAAGGCGCACTTCCCGAGGGAAGGTGTTTTCGTTAGT
AACGGCACGCATTGGTTTGTGACCCAGAGAAATTTTTACGAGCCA
CAGATCATCACAACGGACAACACATTCGTAAGTGGAAACTGTGAT
GTAGTAATTGGCATCGTCAACAATACCGTCTACGACCCTCTCCAA
CCAGAACTCGATTCTTTTAAGGAGGAACTTGATAAATATTTTAAA
AATCATACAAGCCCCGATGTGGACCTCGGAGACATCAGCGGCATT
AATGCGAGCGTGGTCAACATCCAAAAGGAGATAGATCGCCTCAAC
GAAGTTGCCAAGAATTTGAATGAGAGCCTTATaGATCTTCAGGAA
CTCGGCAAGTACGAGCAATACATTAAATGGCCTTGGTACATATGG
CTGGGATTTATTGCAGGGCTGATAGCCATCGTGATGGTGACAATA
ATGCTCTGTTGTATGACTAGTTGTTGCAGCTGCCTTAAAGGGTGC
TGTTCTTGTGGGAGTTGCTGTAAGTTTAAACTTAAGCACACCAAG
AAACGGCAGATAgcgACGGATATTGAAATGAATAGGCTTGGGAAA
taataactcgagtaactaactaacggatccagtgtccagaccatt
gtcttccaaccccagctggcctccagaacacccactggccagtcc
tagagctcctgtccctacccactctttgctacaataaatgctgaa
tgaatccaagatctaaacccttgcggcc.
polyA tail

The present invention provides, in a second aspect, a method of making the EV-based nucleic acid composition, e.g., EV-NAV, of the first aspect of the present invention, the method comprising contacting one or more EVs with the one or more polynucleotides each encoding the S protein or the S protein variant according to the first aspect, and the one or more polynucleotides each encoding the N protein or the N protein variant according to the first aspect, in the presence of a chemical lipofection reagent according to the first aspect, whereby said polynucleotides are loaded into said EVs. In some embodiments, said polynucleotides are pre-mixed with said chemical lipofection reagent. In some embodiments, said polynucleotides are purified prior to being pre-mixed with said chemical lipofection reagent.

The present invention provides, in a third aspect, a method of inducing or eliciting an immune response, or immunizing, against the S protein or the S protein variant in a subject, comprising administering the EV-based nucleic acid composition, e.g., EV-NAV, according to any of claims 1-35 to the subject in an effective amount to produce the immune response. In some embodiments, the immune response comprises a cell-mediated immune response and/or a humoral immune response. In some embodiments, the immune response comprises a T cell response and/or a B cell response. In some embodiments, the immune response is mediated by antibody molecules that are secreted by plasma cells. In some embodiments, the method comprises a single administration of the EV-based nucleic acid composition. In some embodiments, the method further comprises a booster dose of the EV-based nucleic acid composition. In some embodiments, the administering step comprises contacting a muscle tissue of the subject with a device suitable for injection of the composition. In some embodiments, the subject is human. In some embodiments, the subject has previously been infected with SARS-CoV-2. In some embodiments, the subject has not previously been infected with SARS-CoV-2.

### BRIEF DESCRIPTION OF THE DRAWINGS

FIG. 1 schematically illustrates the study design for evaluating the efficacy of the EV-based nucleic vaccine according to the present invention to protect against SARS-CoV-2 infection.

FIG. 2 shows that mice from all groups gained weight and no changes were observed.

FIG. 3 shows quantification of antibodies against SARS-CoV-2 proteins.

FIG. 4 shows representative fluorescence images of HEK293 and HEK293F cells treated with S-2P-VTYA plasmid-lipofectamine and SW1 mRNA-lipofectamine showing Spike protein localization. Top panel shows HEK293 cells treated with S-2P-VTYA plasmid-lipofectamine mixture for 48 hours, fixed with paraformaldehyde and stained with anti-Spike antibody to label Spike protein and Alexa Fluor 647 phalloidin probe to label the cell membrane, and imaged with a Cytation 5 plate reader. Spike protein appears to be localized to the cell membrane (arrow). Bottom panel shows HEK293F cells treated with SW1 mRNA-lipofectamine mixture for 24 hours, fixed with paraformaldehyde and stained with anti-Spike antibody to label Spike protein and Alexa Fluor 647 phalloidin probe to label the cell membrane, and imaged with a Zeiss epifluorescence microscope. Spike protein appears to be localized to the cytoplasm of the cells (dotted arrow) and very little Spike protein is localized to the cell membrane.

FIGS. 5 and 6 show S-potency by flow cytometry for both the SW1 and S-2P-VTYA versions of the SARS-CoV-2 Spike protein.

FIG. 7 shows representative image of Spike mRNA, Nucleocapsid mRNA, and 100 ug/ml input Spike and Nucleocapsid mRNA.

FIG. 8 shows representative dual GFP and DAPI fluorescence images of HEK293F cells treated with Test Formulation: Nucleocapsid expression.

### DETAILED DESCRIPTION

### A. Definitions

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, “and/or” refers to and encompasses any and all possible combinations of one or more of the associated listed items, as well as the lack of combinations when interpreted in the alternative (or).

The terms “about” and “approximate”, as used herein when referring to a measurable value such as an amount, dose, time, temperature, activity, level, number, frequency, percentage, dimension, size, amount, weight, position, length and the like, is meant to encompass variations of ±15%, ±10%, ±5%, ±1%, ±0.5%, or even ±0.1% of the specified amount, dose, time, temperature, activity, level, number, frequency, percentage, dimension, size, amount, weight, position, length and the like. In instances in which the terms “about” and “approximate” are used in connection with the location or position of regions within a reference polypeptide, these terms encompass variations of ±up to 20 amino acid residues, ±up to 15 amino acid residues, ±up to 10 amino acid residues, ±up to 5 amino acid residues, ±up to 4 amino acid residues, ±up to 3 amino acid residues, ±up to 2 amino acid residues, or even ±1 amino acid residue.

The term “derived from” as in “A is derived from B” means that A is obtained from B in such a manner that A is not identical to B.

The terms “treat”, “therapeutic”, “prophylactic” and “prevent” are not intended to be absolute terms. Treatment, prevention and prophylaxis can refer to any delay in onset, amelioration of symptoms, improvement in patient survival, increase in survival time or rate, etc. Treatment, prevention, and prophylaxis can be complete or partial. The term “prophylactic” means not only “prevent”, but also minimize illness and disease. For example, a “prophylactic” agent can be administered to a subject, e.g., a human subject, to prevent infection, or to minimize the extent of illness and disease caused by such infection. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

A treatment can be considered “effective,” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (e.g., progression of the disease is halted). Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. One skilled in the art can monitor efficacy of administration and/or treatment by measuring any one of such parameters, or any combination of parameters.

The term “effective amount” as used herein refers to the amount of a composition or an agent needed to alleviate at least one or more symptom of the disease or disorder, and relates to a sufficient amount of therapeutic composition to provide the desired effect. The term “therapeutically effective amount” refers to an amount of a composition or therapeutic agent that is sufficient to provide a particular effect when administered to a typical subject. An effective amount as used herein, in various contexts, can include an amount sufficient to delay the development of a symptom of the disease, alter the course of a symptom disease (for example but not limited to, slowing the progression of a symptom of the disease), or reverse a symptom of the disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control. The therapeutically effective amount may be administered in one or more doses of the therapeutic agent. The therapeutically effective amount may be administered in a single administration, or over a period of time in a plurality of doses.

“Administering” as used herein can include any suitable routes of administering a therapeutic agent or composition as disclosed herein. Suitable routes of administration include, without limitation, oral, parenteral, intravenous, intramuscular, subcutaneous, transdermal, airway (aerosol), pulmonary, cutaneous, injection or topical administration. Administration can be local or systemic.

As used herein, the term “pharmaceutically acceptable” refers to a carrier that is compatible with the other ingredients of the formulation and not deleterious to the recipient thereof. The term is used synonymously with “physiologically acceptable” and “pharmacologically acceptable”. A pharmaceutical composition will generally comprise agents for buffering and preservation in storage, and can include buffers and carriers for appropriate delivery, depending on the route of administration. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “dose” and “dosage” are used interchangeably herein. A dose refers to the amount of active ingredient given to an individual at each administration. For the present invention, the dose can refer to the concentration of the extracellular vesicles or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The phrase “at least one” includes “a plurality”.

Definitions of common terms in cell biology and molecular biology can be found in “The Merck Manual of Diagnosis and Therapy”, 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

The term “native form” corresponds to the polypeptide as it is understood to be encoded by the infectious agent's genome. The term “exosomal form” corresponds to any derivative of the protein that, in whole or in part, is fused to an exosome-associated protein. The term “cytoplasmic form” corresponds to any derivative of the protein that, in whole or in part, is configured, or designed, to be expressed within the cytoplasm of the cell, rather than entering the canonical secretory pathway.

The expression that a certain protein is “configured, or designed, to be expressed” in a certain way means that its nucleotide sequence encodes certain a particular amino acid sequence such that when that protein is expressed in a cell, that protein will be in its native form, exosomal form, or cytoplasmic form by virtue of that particular amino acid sequence. For instance, if the S protein is expressed in its native form, it is configured, or designed, to induce a humoral immune response by virtue of the fact that it is a transmembrane protein with an extracellular domain. Similarly, the LNL protein is expressed in its exosomal form by virtue of the fact that it is a derivative of the N protein that is in part fused to an exosome-associated protein.

The term “extracellular vesicle” (EV) refers to lipid bilayer-delimited particles that are naturally released from cells. EVs range in diameter from around 20-30 nanometers to about 10 microns or more. EVs can comprise proteins, nucleic acids, lipids and metabolites from the cells that produced them. EVs include exosomes (about 50 to about 100 nm), microvesicles (about 100 to about 300 nm), ectosomes (about 50 to about 1000 nm), apoptotic bodies (about 50 to about 5000 nm) and lipid-protein aggregates of the same dimensions.

The term “chemical lipofection reagent” or “chemical transfection reagent” refers to a cationic-lipid transfection reagent, e.g., Lipofectamine® MessengerMAX™, Lipofectamine® 2000, Lipofectamine® 3000, used to increase the transfection efficiency of RNA (including mRNA and siRNA) or plasmid DNA into in vitro cell cultures by lipofection.

The term “LFA” means lipofectamine.

The term “nucleic acid” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid might be employed for introduction into, e.g., transfection of, cells, e.g., in the form of RNA which can be prepared by in vitro transcription from a DNA template. The RNA can moreover be modified before application by stabilizing sequences, capping, and polyadenylation. Generally, nucleic acid can be extracted, isolated, amplified, or analyzed by a variety of techniques such as those described by, e.g., Green and Sambrook, Molecular Cloning: A Laboratory Manual (Fourth Edition), Cold Spring Harbor Laboratory Press, Woodbury, NY 2,028 pages (2012).

A SARS-CoV-2 virion is approximately 50-200 nanometers in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the complete viral envelope, which are the proteins of interest in regard to the present invention. The spike protein, S, which has been imaged at the atomic level using cryogenic electron microscopy, is the protein responsible for allowing the virus to attach to and fuse with the membrane of a host cell. As used herein, the phrase SARS-CoV-2 structural “protein S, N, M, and/or E” refers to the spike (S), nucleocapsid (N), membrane (M), and/or envelope (E) proteins, respectively, which are encoded by the nucleic acid sequences of the invention, or by a codon-optimized oligonucleotide sequence, encoding each protein individually, or any combination of 2 or 3 proteins, or a combination of all 4 proteins. When two or more nucleic acid sequences are included in a single vector or construct, they are in operable linkage such that the each of the 2, 3, or 4 SARS-CoV-2 structural proteins are properly encoded and expressed. Nucleic acid sequences encoding additional SARS-CoV-2 proteins, such as orfa or orfa/b polypeptides are also included in the nucleic acid sequences of the present invention. Such nucleic acid sequences may be incorporated in a vector as described herein to provide a variation of these vectors.

Cells transfected with a vector as described herein, may be transfected with a vector including a nucleic acid sequence encoding an additional SARS-CoV-2 protein.

The term “S+N” means Spike plus Nucleocapsid.

The term “open reading frame” (ORF) refers to a nucleotide sequence, typically positioned between a start codon and a stop codon, that has the ability to be translated into a polypeptide.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions times 100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

For antibodies, percentage sequence identities can be determined when antibody sequences are maximally aligned by IMGT. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, multiplied by 100 to convert to percentage.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y
where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. The term “nucleic acid sequence” as used herein refers to a sequence of nucleoside or nucleotide monomers consisting of naturally occurring bases, sugars and intersugar (backbone) linkages and includes cDNA. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. It is understood that polynucleotides comprising non-transcribable nucleotide bases may be useful as probes in, for example, hybridization assays. The nucleic acid can be either double stranded or single stranded, and represents the sense or antisense strand. Further, the term “nucleic acid” includes the complementary nucleic acid sequences as well as codon optimized or synonymous codon equivalents.

A variety of host cells are known in the art and suitable for proteins expression and extracellular vesicles production. Non-limiting examples of typical cell used for transfection include, but are not limited to, a bacterial cell, a eukaryotic cell, a yeast cell, an insect cell, or a plant cell. For example, human embryonic kidney 293 (HEK293), E. coli, Bacillus, Streptomyces, Pichia pastoris, Salmonella typhimurium, Drosophila S2, Spodoptera SJ9, CHO, COS (e.g. COS-7), 3T3-F442A, HeLa, HUVEC, HUAEC, NIH 3T3, Jurkat, 293, 293H, or 293F.

A pathogen, which can be a bacteria, virus, or any other microorganism that can cause a disease in a subject, can elicit an immune response (i.e., an integrated bodily response to a pathogen antigen, which can include a cellular immune response and/or a humoral immune response) in the subject. For example, upon contact and/or exposure to a pathogen, a subject may respond with an humoral immune response, characterized by the production of antibody, specifically directed against one or more pathogen antigens.

As used herein the term “antibody” refers to immunoglobulin (Ig) molecules and immunologically active portions of immunoglobulin molecules, i.e., molecules that contain an antigen-binding site that specifically binds an antigen. Antibodies are usually heterotetrameric glycoproteins of about 150,000 Daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known. The antibody may have one or more effector functions which refer to those biological activities attributable to the Fc region (a native sequence Fc region or amino acid sequence variant Fc region or any other modified Fc region) of an antibody. Non-limiting examples of antibody effector functions include Clq binding; complement dependent cytotoxicity; Fc receptor binding; antibody-dependent cell-mediated cytotoxicity (ADCC); phagocytosis; down regulation of cell surface receptors (e.g., B cell receptor (BCR); and cross-presentation of antigens by antigen presenting cells or dendritic cells).

The term “neutralizing antibody” (Nab) refers an antibody that defends a cell from a pathogen or infectious particle by neutralizing any effect it has biologically. Neutralization renders the particle no longer infectious or pathogenic. Neutralizing antibodies are part of the humoral response of the adaptive immune system against viruses, intracellular bacteria and microbial toxin. By binding specifically to surface antigen on an infectious particle, neutralizing antibodies prevent the particle from interacting with its host cells it might infect and destroy. Immunity due to neutralizing antibodies is also known as sterilizing immunity, as the immune system eliminates the infectious particle before any infection took place.

The term “antigen” refers to any substance that will elicit an immune response. For instance, an antigen relates to any substance, preferably a peptide or protein, that reacts specifically with antibodies or T-lymphocytes (T cells). As used herein, the term “antigen” comprises any molecule which comprises at least one epitope. For instance, an antigen is a molecule which, optionally after processing, induces an immune reaction. For instance, any suitable antigen may be used, which is a candidate for an immune reaction, wherein the immune reaction may be a cellular immune reaction. For instance, the antigen may be presented by a cell, which results in an immune reaction against the antigen. For example, an antigen is a product which corresponds to or is derived from a naturally occurring antigen. Such antigens include, but are not limited to, SARS-CoV-2 structural proteins S, N, M, and E, and any variants or mutants thereof.

The terms “peptide”, “polypeptide” and “protein” are used interchangeably herein and refer to any chain of at least two amino acids, linked by a covalent chemical bound. As used herein a peptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

The term “pharmaceutical composition” refers to a formulation comprising an active ingredient, and optionally a pharmaceutically acceptable carrier, diluent or excipient. The term “active ingredient” can interchangeably refer to an “effective ingredient,” and is meant to refer to any agent that is capable of inducing a sought-after effect upon administration. By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

The term “vaccine” relates to a pharmaceutical preparation (pharmaceutical composition) or product that upon administration induces an immune response, e.g., a cellular immune response, which recognizes and attacks a pathogen or a diseased cell. The term “immune response” refers to an integrated bodily response to an antigen and refers to a cellular immune response and/or a humoral immune response. The immune response may be protective/preventive/prophylactic and/or therapeutic.

The term “cellular immune response” or “cell-mediated immune response” describes any adaptive immune response in which antigen-specific T cells have the main role. It is defined operationally as all adaptive immunity that cannot be transferred to a native recipient by serum antibody. In contrast, the term “humoral immune response” describes immunity due to antibodies.

The cellular response relates to cells called T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells.

The terms “immunoreactive cell” “immune cells” or “immune effector cells” relate to a cell which exerts effector functions during an immune reaction. An “immunoreactive cell” preferably is capable of binding an antigen or a cell characterized by presentation of an antigen or an antigen peptide derived from an antigen and mediating an immune response. For example, such cells secrete cytokines and/or chemokines, secrete antibodies, recognize cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells.

The term “adjuvant” refers to a pharmacological or immunological agent that modifies the effect of other agents. An adjuvant may be added to the vaccine composition of the invention to boost the immune response to produce more antibodies and longer-lasting immunity, thus minimizing the dose of antigen needed. Adjuvants may also be used to enhance the efficacy of a vaccine by helping to modify the immune response to particular types of immune system cells: for example, by activating T cells instead of antibody-secreting B cells depending on the purpose of the vaccine. Immunologic adjuvants are added to vaccines to stimulate the immune system's response to the target antigen, but do not provide immunity themselves. Examples of adjuvants include, but are not limited to analgesic adjuvants; inorganic compounds such as alum, aluminum hydroxide, aluminum phosphate, calcium phosphate hydroxide; mineral oil such as paraffin oil; bacterial products such as killed bacteria (Bordetella pertussis, Mycobacterium bovis, toxoids); nonbacterial organics such as squalene; delivery systems such as detergents (Quil A); plant saponins from Quillaja, soybean, or Polygala senega; cytokines such as IL-1, IL-2, IL-12; combination such as Freund's complete adjuvant, Freund's incomplete adjuvant; food-based oil such as Adjuvant 65, which is based on peanut oil.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### B. Cardiospheres

Cardiospheres are undifferentiated cardiac cells that grow as self-adherent clusters as described in WO 2005/012510, and Messina et al., “Isolation and Expansion of Adult Cardiac Stem Cells from Human and Murine Heart,” Circulation Research, 95:911-921 (2004), the disclosures of which are herein incorporated by reference in their entirety.

Briefly, heart tissue can be collected from a patient during surgery or cardiac biopsy. The heart tissue can be harvested from the left ventricle, right ventricle, septum, left atrium, right atrium, Crista terminalis, right ventricular endocardium, septal or ventricle wall, atrial appendages, or combinations thereof. A biopsy can be obtained, e.g., by using a percutaneous bioptome as described in, e.g., U.S. Patent Application Publication Nos. 2009/012422 and 2012/0039857, the disclosures of which are herein incorporated by reference in their entirety.

The tissue can then be cultured directly, or alternatively, the heart tissue can be frozen, thawed, and then cultured. The tissue can be digested with protease enzymes such as collagenase, trypsin and the like. The heart tissue can be cultured as an explant such that cells including fibroblast-like cells and cardiosphere forming cells grow out from the explant. In some instances, an explant is cultured on a culture vessel coated with one or more components of the extracellular matrix (e.g., fibronectin, laminin, collagen, elastin, or other extracellular matrix proteins). The tissue explant can be cultured for about 1, 2, 3, 4, or more weeks prior to collecting the cardiosphere-forming cells. A layer of fibroblast-like cells can grow from the explant onto which cardiosphere-forming cells appear.

Cardiosphere-forming cells can appear as small, round, phase-bright cells under phase contrast microscopy. Cells surrounding the explant including cardiosphere-forming cells can be collected by manual methods or by enzymatic digestion. The collected cardiosphere-forming cells can be cultured under conditions to promote the formation of cardiospheres. In some aspects, the cells are cultured in cardiosphere-growth medium comprising buffered media, amino acids, nutrients, serum or serum replacement, growth factors including but not limited to EGF and bFGF, cytokines including but not limited to cardiotrophin, and other cardiosphere promoting factors such as but not limited to thrombin. Cardiosphere-forming cells can be plated at an appropriate density necessary for cardiosphere formation, such as about 20,000-100,000 cells/mL. The cells can be cultured on sterile dishes coated with poly-D-lysine, or other natural or synthetic molecules that hinder the cells from attaching to the surface of the dish. Cardiospheres can appear spontaneously about 2-7 days or more after cardiosphere-forming cells are plated.

### C. Cardiosphere-Derived Cells (CDCs)

CDCs are a population of cells generated by manipulating cardiospheres in the manner as described in, e.g., U.S. Patent Application Publication No. 2012/0315252, the disclosures of which are herein incorporated by reference in their entirety. For example, CDCs can be generated by plating cardiospheres on a solid surface which is coated with a substance which encourages adherence of cells to a solid surface of a culture vessel, e.g., fibronectin, a hydrogel, a polymer, laminin, serum, collagen, gelatin, or poly-D-lysine, and expanding same as an adherent monolayer culture. CDCs can be repeatedly passaged, e.g., passaged two times or more, according to standard cell culturing methods.

D. Activated-specialized tissue-effector cells (ASTECs) and ASTEC-derived exosomes (ASTEX) ASTECs and ASTEX are activation-specialized tissue-effector cells and EVs, e.g, exosomes, derived therefrom, respectively, as described in WO 2019/152409, and Ibrahim et al., Augmenting canonical Wnt signalling in therapeutically inert cells converts them into therapeutically potent exosome factories, Nat Biomed Eng., 2019 September; 3(9):695-705, the disclosures of which are herein incorporated by reference in their entirety.

### E. Exosomes

Exosomes are defined herein as all small, secreted vesicles of ˜20-150 nm that are released by mammalian cells, and made either by budding into endosomes or by budding from the plasma membrane of a cell. Exosomes can range in size from approximately 20-150 nm in diameter. In some cases, they have a characteristic buoyant density of approximately 1.1-1.2 g/mL, and a characteristic lipid composition. Their lipid membrane is typically rich in cholesterol and contains sphingomyelin, ceramide, lipid rafts and exposed phosphatidylserine. Exosomes express certain marker proteins, such as integrins and cell adhesion molecules, but generally lack markers of lysosomes, mitochondria, or caveolae. In some embodiments, the exosomes contain cell-derived components, such as but not limited to, proteins, DNA and RNA (e.g., microRNA and noncoding RNA). In some embodiments, exosomes can be obtained from cells obtained from a source that is allogeneic, autologous, xenogeneic, or syngeneic with respect to the recipient of the exosomes.

Certain types of RNA, e.g., microRNA (miRNA), are known to be carried by exosomes.

miRNAs function as post-transcriptional regulators, often through binding to complementary sequences on target messenger RNA transcripts (mRNAs), thereby resulting in translational repression, target mRNA degradation and/or gene silencing. For example, as described in WO/2014/028493, miR146a exhibits over a 250-fold increased expression in CDCs, and miR210 is upregulated approximately 30-fold, as compared to the exosomes isolated from normal human dermal fibroblasts.

Exosomes derived from cardiospheres and CDCs are described in, e.g., WO/2014/028493, the disclosures of which are herein incorporated by reference in their entirety. Methods for preparing exosomes can include the steps of: culturing cardiospheres or CDCs in conditioned media, isolating the cells from the conditioned media, purifying the exosome by, e.g., sequential centrifugation, and optionally, clarifying the exosomes on a density gradient, e.g., sucrose density gradient. In some instances, the isolated and purified exosomes are essentially free of non-exosome components, such as components of cardiospheres or CDCs. Exosomes can be resuspended in a buffer such as a sterile PBS buffer containing 0.01-1% human serum albumin. The exosomes may be frozen and stored for future use.

Exosomes can be collected, concentrated and/or purified using methods known in the art. For example, differential centrifugation has become a leading technique wherein secreted exosomes are isolated from the supernatants of cultured cells. This approach allows for separation of exosomes from larger extracellular vesicles and from most non-particulate contaminants by exploiting their size. Exosomes can be prepared as described in a wide array of papers, including but not limited to, Fordjour et al., “A shared pathway of exosome biogenesis operates at plasma and endosome membranes”, bioRxiv, preprint posted Feb. 11, 2019, at https://www.biorxiv.org/content/10.1101/545228v1; Booth et al., “Exosomes and HIV Gag bud from endosome-like domains of the T cell plasma membrane”, J Cell Biol., 172:923-935 (2006); and, Fang et al., “Higher-order oligomerization targets plasma membrane proteins and HIV gag to exosomes”, PLoS Biol., 5:e158 (2007). Exosomes using a commercial kit such as, but not limited to the ExoSpin™ Exosome Purification Kit, Invitrogen® Total Exosome Purification Kit, PureExo® Exosome Isolation Kit, and ExoCap™ Exosome Isolation kit. Methods for isolating exosome from stem cells are found in, e.g., Tan et al., Journal of Extracellular Vesicles, 2:22614 (2013); Ono et al., Sci Signal, 7(332):ra63 (2014) and U.S. Application Publication Nos. 2012/0093885 and 2014/0004601. Methods for isolating exosome from cardiosphere-derived cells are found in, e.g., Ibrahim et al., Exosomes as critical agents of cardiac regeneration triggered by cell therapy, Stem Cell Reports, 2014. Specific methodologies include ultracentrifugation, density gradient, HPLC, adherence to substrate based on affinity, or filtration based on size exclusion.

Size exclusion allows for their separation from biochemically similar, but biophysically different microvesicles, which possess larger diameters of up to 1,000 nm. Differences in flotation velocity further allows for separation of differentially sized exosomes. In general, exosome sizes will possess a diameter ranging from 30-200 nm, including sizes of 4

0-100 nm. Further purification may rely on specific properties of the particular exosomes of interest. This includes, e.g., use of immunoadsorption with a protein of interest to select specific vesicles with exoplasmic or outward orientations.

Among current methods, e.g., differential centrifugation, discontinuous density gradients, immunoaffinity, ultrafiltration and high performance liquid chromatography (HPLC), differential ultracentrifugation is the most commonly used for exosome isolation. This technique utilizes increasing centrifugal force from 2000×g to 10,000×g to separate the medium- and larger-sized particles and cell debris from the exosome pellet at 100,000×g. Centrifugation alone allows for significant separation/collection of exosomes from a conditioned medium, although it is insufficient to remove various protein aggregates, genetic materials, particulates from media and cell debris that are common contaminants. Enhanced specificity of exosome purification may deploy sequential centrifugation in combination with ultrafiltration, or equilibrium density gradient centrifugation in a sucrose density gradient, to provide for the greater purity of the exosome preparation (flotation density 1.1-1.2 g/mL) or application of a discrete sugar cushion in preparation.

Importantly, ultrafiltration can be used to purify exosomes without compromising their biological activity. Membranes with different pore sizes—such as 100 kDa molecular weight cutoff (MWCO) and gel filtration to eliminate smaller particles—have been used to avoid the use of a nonneutral pH or non-physiological salt concentration. Currently available tangential flow filtration (TFF) systems are scalable (to >10,000 L), allowing one to not only purify, but concentrate the exosome fractions, and such approaches are less time consuming than differential centrifugation. HPLC can also be used to purify exosomes to more uniformly sized particle preparations and preserve their biological activity as the preparation is maintained at a physiological pH and salt concentration. Other chemical methods have exploit differential solubility of exosomes for precipitation techniques, addition to volume-excluding polymers (e.g., polyethylene glycols (PEGs)), possibly combined additional rounds of centrifugation or filtration. For example, a precipitation reagent, ExoQuick®, can be added to conditioned cell media to quickly and rapidly precipitate a population of exosomes, although re-suspension of pellets prepared via this technique may be difficult. Flow field-flow fractionation (FlFFF) is an elution-based technique that is used to separate and characterize macromolecules (e.g., proteins) and nano- to micro-sized particles (e.g., organelles and cells) and which has been successfully applied to fractionate exosomes from culture media.

Beyond these techniques relying on general biochemical and biophysical features, focused techniques may be applied to isolate specific exosomes of interest. This includes relying on antibody immunoaffinity to recognizing certain exosome-associated antigens. As described, exosomes further express the extracellular domain of membrane-bound receptors at the surface of the membrane. This presents a ripe opportunity for isolating and segregating exosomes in connections with their parental cellular origin, based on a shared antigenic profile. Conjugation to magnetic beads, chromatography matrices, plates or microfluidic devices allows isolating of specific exosome populations of interest as may be related to their production from a parent cell of interest or associated cellular regulatory state. Other affinity-capture methods use lectins which bind to specific saccharide residues on the exosome surface.

F. 10 KDa & 1000 KDa Method CDC-EV (10 KDa 5 or 1000 KDa) drug substance is obtained after filtering CDC conditioned medium (CM) containing EVs through a 10 KDa or 1000 KDa pore size filter, wherein the final product, composed of secreted EVs and concentrated CM, is formulated in PlasmaLyte A by diafiltration and stored frozen.

### G. MSC-EVs

EVs originating from human bone marrow mesenchymal stem cells (MSC-EVs) are obtained after filtering MSC CM containing EVs through a 10 KDa pore size filter following a similar process as for CDC-EV production. MSC-EVs are a non-cellular, filter sterilized product obtained from human MSCs cultured under defined, serum-free conditions. The final product, composed of secreted EVs and concentrated CM, is formulated in PlasmaLyte A and stored frozen. The frozen final product is “ready to use” for direct subconjunctival injection after thawing.

### H. Examples

It will be appreciated by persons skilled in the art that numerous variations and/or modifications may be made to the present invention as shown in the specific embodiments without departing from the spirit or scope of the present invention as broadly described. The present embodiments are, therefore, to be considered in all respects as illustrative and not restrictive. In order that the present invention may be readily understood and put into practical effect, particular preferred embodiments will now be described by way of the following non-limiting examples.

### Cell Culture and Exosome Preparation.

293F cells (ThermoFisher) were maintained in FreeStyle 293 Expression Medium (Gibco, #12338-018) and incubated at 37° C. in 8% C02. For exosome production, 293F cells were seeded at a density of 1.5×10\hat{ }6 cells/ml in shaker flasks in a volume of ˜¼ the flask volume and grown at a shaking speed of 110 rpm. The exosome-containing media was sterilized by gravity vacuum sterile filtration through a 220 nm pore-size diameter filter into collection bottle, generating a ‘clarified tissue culture supernatant’ (CTCS); this step removes remaining cells, cell fragments, and vesicles larger than 220 nm. The CTCS was subjected to centrifugal filtration against a 100 kDa pore size membrane, generating a ‘concentrated vesicle suspension’ (CVS) containing exosomes. The CVS was subjected to size exclusion chromatography (SEC) to separate exosomes. Obtained exosome-containing fractions are pooled and concentrated by centrifugal filtration, generating a purified exosome preparation (PE). PE were quantified using the NTA, diluted to a concentration of 1×10 particles/mL and stored at −80° C., until use.

### Formulation of EV-Based Nucleic Acid Composition.

Components of formulation are listed in Table 1 below. For formulation preparation, PE were thawed on ice and diluted at working concentration of 1×10\hat{ }11 particles/mL. S-2P-VTYA mRNA and LNL mRNA were thawed on ice, and concentration, integrity and purity verified at the Bioanalyzer (Agilent). Lipofectamine MessengerMax was acquired from Invitrogen/Thermofisher. The formulation was prepared in a biological safety cabinet. Lipofectamine was combined with MEM, gently mixed, and incubated for 10 minutes at RT. S-2P-VTYA mRNA and LNL mRNA (for a total of 100 ug/mL) were mixed with MEM, gently mixed, and incubated for 10 minutes at RT. The two solutions were subsequently mixed and incubated for 5 minutes at RT to initiate lipid-coating of the mRNA molecules. Lastly, the exosomes were added to the mix, gently mixed, and incubated for 10 min at RT. After incubation, the formulation was transferred to ice, aliquoted as needed and stored at −80° C. until use. For control group, receiving exosome only, and LFA+mRNA group, volume of excluded components was replaced by PBS (Table 2).

TABLE 1
Test Formulation: components and concentrations

| Components | Stock | Control (exosome only) | LFA + mRNA | |
|---|---|---|---|---|
| S-2P-VTYA mRNA | 1 mg/mL | Replaced by equivalent | 50 | μg |
| | | volume of PBS | | |
| LNL mRNA | 1 mg/mL | Replaced by equivalent | 50 | μg |
| | | volume of PBS | | |
| Lipofectamine | vendor | Replaced by equivalent | 200 | μL |
| MessengerMax | | volume of PBS | | |
| Exosomes | 1 × 10\hat{ }11 | 300 μL (3 × 10\hat{ }10 exosomes) | Replaced by | |
| | exosomes/mL | | equivalent volume | |
| | | | of PBS | |
| MEM | | 400 μL | 400 | μL |
| Total | | 1000 μL | 1000 | μL |

TABLE 2
Control formulations

| Components | Stock | Clinical batch | |
|---|---|---|---|
| S-2P-VTYA mRNA | 1 mg/mL | 50 | μg |
| LNL mRNA | 1 mg/mL | 50 | μg |
| Lipofectamine | vendor | 200 | μL |
| MessengerMax | | | |
| Exosomes | 1 × 10\hat{ }11 | 300 μL (3 × 10\hat{ }10 | |
| | exosomes/mL | exosomes) | |
| MEM | | 400 | μL |
| Total | | 1000 | μL |

### Animal Study.

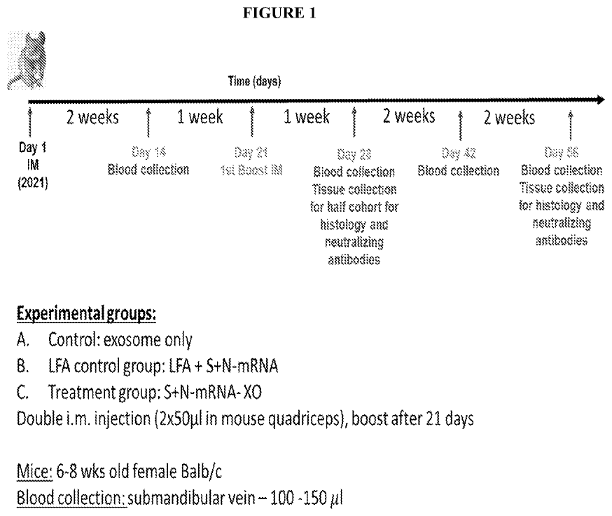

To test the efficacy of exosomes loaded with S-2P-VTYA mRNA and LNL mRNA (“Test Formulation”), age-matched BALB/c mice (female, 6-8 wks old) were anesthetized using isoflurane and received two bilateral intramuscular injection (50 ul per leg, total 100 ul, for a final concentration of 10 ug mRNA) spaced 21 days. The mice were divided in three groups as shown in FIG. 1.

Group A was used as a control, and received exosome at a final concentration of 3×10\hat{ }10 particles per mL.

Group B received formulation containing LFA and S-2P-VTYA+LNL mRNA only (final 10 μg mRNA/mouse).

Group C received full Test Formulation (S+N-mRNA XO, final 10 μg mRNA/mouse).

Booster injection was performed at day 21. Mice were monitored closely for changes in health, weight recorded biweekly. Colony was divided in two groups, to allow tissue and blood collection at two time-points: 28 days and 56 days. At each time-point, mice were anesthetized using isoflurane, euthanized by cervical dislocation, and organs collected for histology analysis. In brief, brain, salivary glands, lungs, heart, diaphragm, liver, intestine, kidneys and spleen were collected, fixed in 10% neutral buffered formalin and sent for analysis to the HIC/Comparative Pathology Program of the University of Washington. A section of the spleen was retained for splenocytes isolation and in vitro analysis of the cellular immune response. Blood (˜500 ul) was collected from the submandibular vein and processed for plasma isolation after centrifugation at 4000 rpm for 5 min at 4° C.

### ELISA for SARS-CoV-2 Antigen-Specific Antibody Responses.

Mouse IgG antibody against SARS-CoV-2 antigens was measured by enzyme-linked immunosorbent assays (ELISA) using precoated ELISA plates (IEQ-CoV-S-RBD-IgG and IEQ-CoVN-IgG, RayBiotech) according to the manufacturer's instructions, at RT. Briefly, mouse plasmas were diluted 1:50 in sample buffer (RayBiotech) and added to antigen-coated wells, in triplicates, and incubated at room temperature (RT) for 2 hours on a shaker (200 rpm). Commercially available antibody against either Spike or Nucleocapsid were used as positive controls. Plates were washed 3 times with washing buffer, incubated for 2 hours at RT with HRP-conjugated goat anti-mouse secondary antibodies (1:5000, Jackson ImmunoResearch) diluted in assay buffer (RayBiotech). After 3 washes, plates were developed using TMB substrate (RayBiotech). Absorbance at 650 nm was recorded at time 0 and every 2 min afterwards, up to 30 min using a BioTeck Gen5 plate reader (Agilent).

### Neutralizing Antibodies.

Plasma samples will be heat-inactivated at 56° C. for 30 min and sent to RetroVirox, Inc for quantification of neutralizing antibodies.

### Spleenocyte Isolation.

Spleens were processed for single cell isolation by mechanical disruption of spleen pouch using a syringe stopper and passage through a 0.040 mm mesh size nylon cell strainer to remove tissue debris. Erythrocytes were lysed using ammonium chloride potassium (ACK) buffer (ThermoFisher), splenocytes were collected by centrifugation at 300×g for 5 minutes. Cellular pellet was resuspended in completed RPMI 1640 media (ATCC).

### Proliferation Assay.

Splenocytes will be resuspended in 10% FBS in PBS at a concentration of 2×10\hat{ }6 cells/mL and incubated with 10 ug/mL of carboxyfluorescein succinimidyl ester (CFSE) (ThermoFisher) for 15 min at 37° C. Cells will be subsequently washed with complete media and incubated for 96 hours in complete media (baseline) or in the presence of 10 ug/ul of SARS-CoV-2 antigens S (SIN-C52H4, Acro Biosystems) or N (NUN-C5227, Acro Biosystems). After 96 hours, cells will be washed with 0.2 mL 10% FBS in PBS and centrifuged at 300×g for 5 minutes at RT. Cells will be then stained with anti-CD3-APC (ThermoFisher, #17-0032-82), anti-CD4-PerCP-Cy5.5 (Biolegend, #100433), or anti-CD8-PE antibodies (Biolegend, #MCD0801) for 30 minutes at 4° C. The stained cells were washed twice with 0.2 mL PBS, fixed and analyzed by flow cytometry using a flow cytometer (FACS Canto II, BD Biosciences). Cells will be gated first for CD3+ T-cells and then for CD4+/CD8− or CD8+/CD4− populations. Data will be analyzed using FlowJo 10 software (FlowJo LLC).

### ELISPOT.

To evaluate the cellular immune response, splenocytes will be plated at a concentration of 5×10\hat{ }5 cells/well and incubated for 24 h. Commercially available ELISPOT plate for evaluation of IL4, INFg and IgG will be used. Assay will follow manufacture's guidelines.

### Statistical Analysis.

Data were analyzed using Excel and GraphPad Prism 9.1 and shown as mean±sem. 1-way ANOVA with post-hoc correction for multiple comparisons or 2-tailed t-test were applied as needed.

### Animal Study.

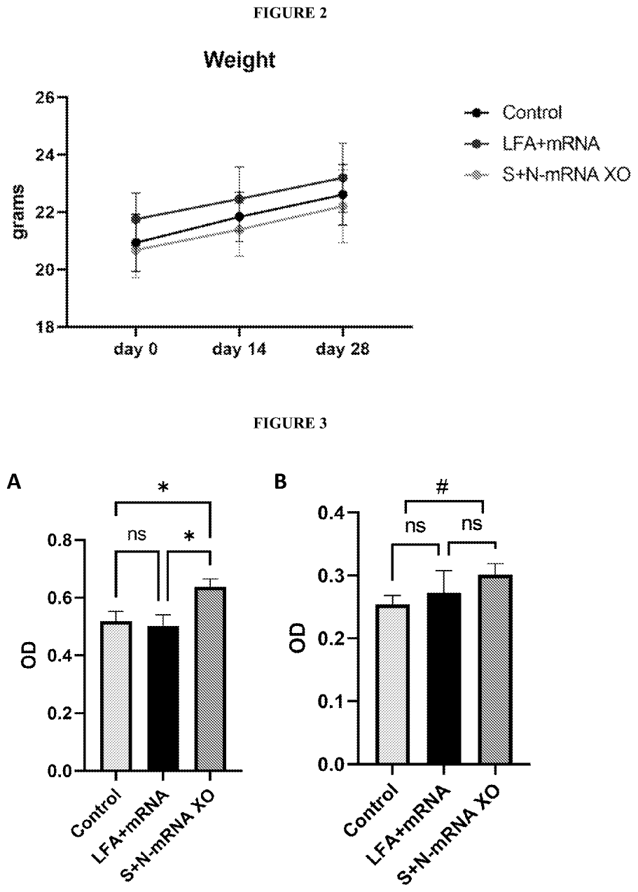

Health and weight were closely monitored. Referring to FIG. 2, no changes in weight were observed up to one week following the 2 separate injections. Data are shown as mean±SD.

Test Formulation induced the production of antibodies against SARS-CoV-2 proteins.

ELISA kits from RayBiotech were modified to allow detection of mouse antibodies (IgG). It was observed observed that mice receiving Test Formulation showed a 25% increase of IgG against SARS-CoV-2 Spike (p<0.05, ANOVA) and 20% increase of IgG against SARS-CoV-2 Nucleocapsid (p<0.05, 2-tailed t-test vs ctrl), while no increase over control background was observed for LFA+mRNA. Same quantification will be performed at day 56 and increase in immunoresponse and production of SARS-CoV-2 IgG is anticipated.

Referring to FIG. 3, Test Formulation induced a 25% increase of IgG against SARS-CoV-2 Spike (A, p<0.05, ANOVA) and 20% increase of IgG against SARS-CoV-2 Nucleocapsid (B, p<0.05, 2-tailed t-test vs ctrl), while no increase over control background was observed for LFA+mRNA. Data are shown as mean±SEM. *p<0.05, ns=not significant, 1-way ANOVA; #p<0.05, 2-tailed t-test vs control.

### Cellular Immune Response.

Cellular immune response will be evaluate using primary splenocytes isolated from control and treated mice. Using ELISPOT technology, the Th immune response will be tested, evaluating the expression of interferon gamma (IFN-7) and interleukin 4 (IL-4). A proliferation assay will be used to identify what cellular response is induced by the viral proteins. Cells will be cultured in w/o w/o either Spike or Nucleocapsid protein: quantification of cell proliferation induced by the viral proteins will clarify the effect of vaccination on CD4+ T-cells and/or CD8+ T cells, and establish the extent of immunological memory induced by the vaccination.

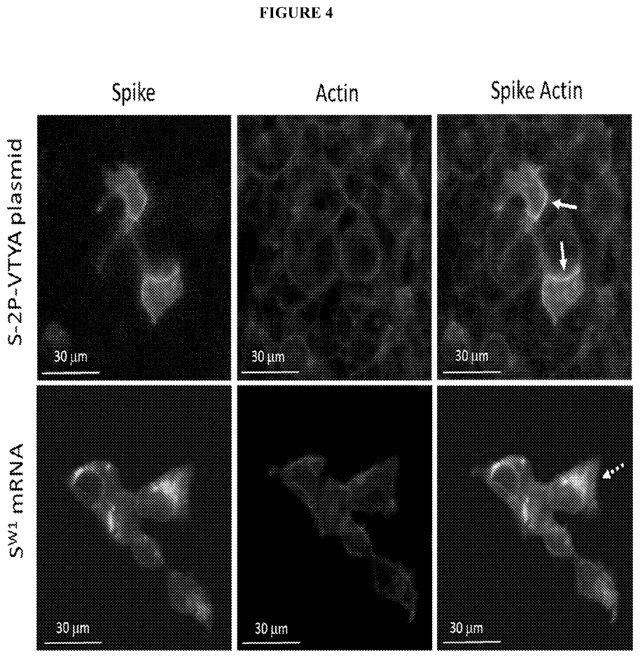

S-2P-VTYA plasmid image (top panel of FIG. 4).

10,000 HEK293 cells in 150 μL of growth media were plated in tissue culture treated 96 well plates and grown for 24 hours. Spike (S-2P-VTYA) plasmid was thawed on ice. 0.2 μg of S-2P-VTYA plasmid were coated with cationic lipid (Lipofectamine 3000 reagent). HEK293 cells were treated with 10 μL of S-2P-VTYA plasmid-lipofectamine mixture and grown for 48 hours. Cells were fixed with paraformaldehyde and stained with anti-Spike antibody to label Spike protein and Alexa Fluor 647 phalloidin probe to label the cell membrane. Fluorescence images were acquired using a Cytation 5 plate reader.

SW1 mRNA image (bottom panel of FIG. 4).

100,000 HEK293F cells in 1 mL of growth media were plated on Poly-L-Lysine coverslips in a 12 well plate and grown for 24 hours. Spike (Sw1) mRNA was thawed on ice. 5 μg of Sw1 mRNA were coated with cationic lipid (Lipofectamine Messenger Max). HEK293F cells were treated with 108 μL of Sw1 mRNA-lipofectamine mixture for 24 hours. Cells were fixed with paraformaldehyde and stained with anti-Spike antibody to label Spike protein and Alexa Fluor 647 phalloidin probe to label the cell membrane. Fluorescence images were acquired using a Zeiss epifluorescence microscope.

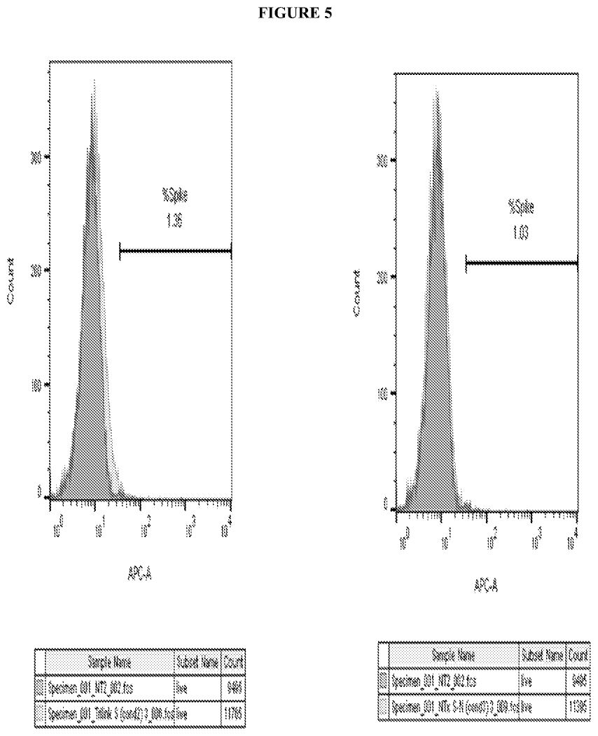

Potency Assay #1 (FIG. 5; Table 3)

### Cell Seeding (Day 1)

HEK293F cells were cultured to 80-90% confluency, trypsinized, and counted. The cells were seeded in 6-well plates at a density of 150,000 cells/mL. The cells were then allowed to attach and grow overnight under standard cell culture conditions

### Cell Treatment (Day 2)

Test Formulations were thawed on ice. The media in the 6-well dishes was aspirated and replaced with new complete DMEM (37° C.). The cells were treated with 120 μL of formulations per well (6 μg S mRNA per well). The plate was gently rocked to mix the formulation and returned cultured under normal conditions for 24 hours.

### Analysis by Flow Cytometry (day 3)

Prior to analysis the anti-spike antibody (ab273433) was labeled with AlexaFluor647 using a kit (ab269823). Following the manufactures recommended protocol, 10 μL of modifier solution was mixed with 100 μL of antibody; this mixture was then added to the lyophilized AlexaFluor647 powder included in the kit. Following 15 minutes incubation at room temperature 10 μL of quencher solution was added to the mixture.

The transfected cells were visualized for viability. The cells were detached using TrypLE select and counted. 250,000 cells were aliquoted into new tubes and washed three times with PBS at room temperature, 300×g, for 5 minutes. Each condition was incubated with 1.5 μL of labeled antibody for 30 minutes at room temperature in the dark. Following incubation, the cells were washed three times with PBS at room temperature, 300×g, for 5 minutes. Next, the cells were incubated with calcein AM dye (BD 564061) for 15 minutes at room temperature in the dark. A BD FACSCantoII was used to measure staining.

### Statistical Analysis

The data was exported to FlowJo for further analysis. Live cells were gated based on positive calcein AM (FITC) staining. The live cells were next gated for spike (APC) staining by setting the non-treated cells to 0.5-1% signal. The results were copied into Microsoft Excel where the average and standard deviations were calculated.

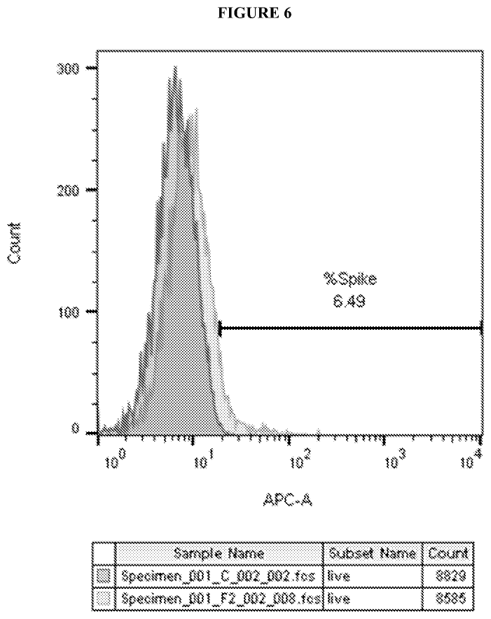

Potency Assay #2 (FIG. 6; Table 3)

### Cell Seeding (Day 1)

HEK293 cells were cultured to 80-90% confluency, trypsinized, and counted. The cells were seeded in 6-well plates at a density of 150,000 cells/mL. The cells were then allowed to attach and grow overnight under standard cell culture conditions.

### Cell Treatment (Day 2)

Test Formulations were thawed on ice. The media in the 6-well dishes was aspirated and replaced with new complete DMEM (37° C.). The cells were treated with 120 μL of formulations per well (6 μg S2P-VTYA mRNA per well). The plate was gently rocked to mix the formulation and returned cultured under normal conditions for 24 hours.

### Analysis by Flow Cytometry (Day 3)

Prior to analysis the anti-spike antibody (ab273433) was labeled with AlexaFluor647 using a kit (ab269823). Following the manufactures recommended protocol, 10 μL of modifier solution was mixed with 100 μL of antibody; this mixture was then added to the lyophilized AlexaFluor647 powder included in the kit. Following 15 minutes incubation at room temperature 10 μL of quencher solution was added to the mixture.

The transfected cells were visualized for viability. The cells were detached using TrypLE express and counted. 250,000 cells were aliquoted into new tubes and washed three times with PBS at room temperature, 300×g, for 5 minutes. Each condition was incubated with 1.5 μL of labeled antibody for 30 minutes at room temperature in the dark. Following incubation, the cells were washed three times with PBS at room temperature, 300×g, for 5 minutes. Next, the cells were incubated with calcein AM dye (BD 564061) for 15 minutes at room temperature in the dark. A BD FACSCantoII was used to measure staining.

### Statistical Analysis

The data was exported to FlowJo for further analysis. Live cells were gated based on positive calcein AM (FITC) staining. The live cells were next gated for spike (APC) staining by setting the non-treated cells to 0.5-1% signal. The results were copied into Microsoft Excel where the average and standard deviations were calculated.

TABLE 3

| | average % Spike | standard deviation |
|---|---|---|
| Control (Potency Assay #1) | 0.77 | 0.38 |
| S (Potency Assay #1) | 1.34 | 0.07 |
| S (Potency Assay #1) | 1.01 | 0.13 |
| Control (Potency Assay #2) | 0.61 | 0.16 |
| S2P-VTYA (Potency Assay #2) | 5.81 | 0.83 |

### Potency Assay: Immunofluorescence for Nucleocapsid.

Cells were examined under phase objective to ensure health growth. The growth media was carefully aspirated from the wells and cells were wash with PBS to remove cell debris. Cells were fixed with 4% PFA for 15 minutes. Cells were washed with PBS for 5 minutes (2 times) and then blocked with blocking buffer for 1 hour. Cells were immunostained with 1:100 dilution anti-Nucleocapsid antibody (HL-455) in antibody dilution buffer for 2 hours. Cells were washed with PBS for 5 minutes (2 times) and then incubated with 1:100 dilution anti-mouse Alexafluor 488 antibody in antibody dilution buffer for 1 hour. Cells were washed with PBS for 5 minutes (2 times). Cells were incubated with 1:1000 DAPI solution in PBS for 5 minutes. Cells were then washed with PBS for 5 minutes. All steps were performed at RT. Representative images were acquired from the GFP and DAPI channels and at 10× and 20× magnification. Images were acquired from the center of the wells. The exposure settings were identical for each condition. Raw images were preprocessed using rolling ball subtraction and analyzed to obtain the number of DAPI cells and the number of GFP positive DAPI cells.

Potency assay for Test formulation showed sustained expression of SARS-CoV-2 proteins: Spike.

TABLE 4
Summary of potency assay of Test Formulation.

| | % Spike by | % Spike | % Nucleocapsid |
|---|---|---|---|
| Condition | Flow Cytometry | by IF | by IF |
| S + N Test | 5.8% | 28.88 | 15.43 |
| Formulation | | | |
| day 1 | | | |
| S + N Test | 4.4 | 11.09 | 16.45 |
| Formulation | | | |
| day 21 | | | |

Spike and Nucleocapsid mRNA Preparation.

Spike and Nucleocapsid mRNA were obtained at a concentration of 2 mg/mL from Trilink Biotechnologies. mRNAs were diluted with ultrapure distilled water to a concentration of 1 mg/mL using RNase-free tips and tubes.

mRNA Integrity.

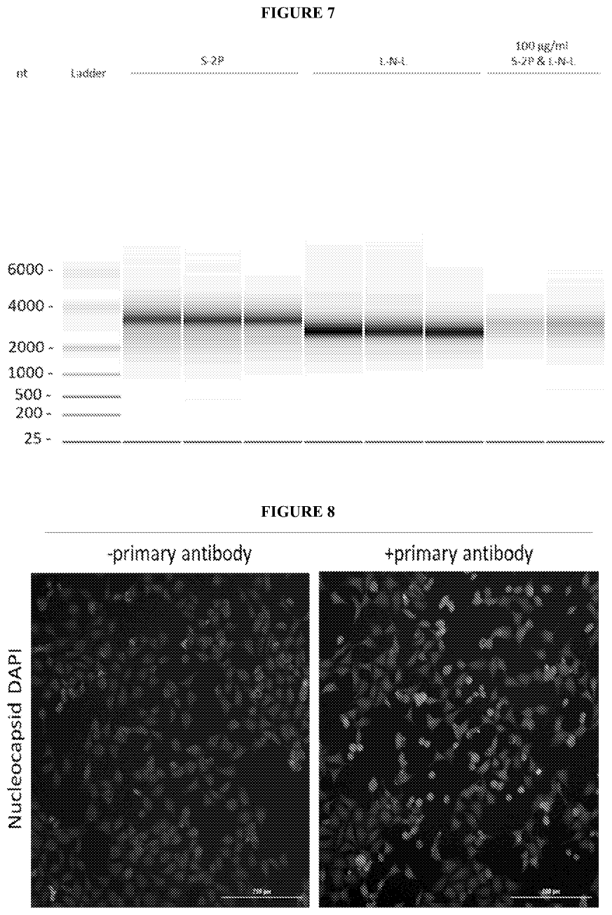

Spike and Nucleocapsid mRNA were analyzed on the Agilent 2100 Bioanalyzer using the RNA Nanochip (FIG. 7). Spike mRNA alone has a size of about 3800 nucleotides and Nucleocapsid mRNA alone has a size of about 2800 nucleotides which was consistent with respective mRNA release specifications. When mixed, Spike and Nucleocapsid mRNA was observed as two bands that are close to one another. This is expected since the Spike and Nucleocapsid mRNA alone are 1000 nucleotides apart, resulting in two close bands.

### High Encapsulation Efficiency for Test Formulation.

Table 4 displays the mRNA encapsulation efficiency of Test Formulation. The mRNA standard curve exhibited a strong linear relationship between concentration and fluorescence intensity with an R2 of 0.99. 83.04%±11.85% of Spike and Nucleocapsid mRNA was encapsulated into Test Formulation (Table 5), which equates to 16.96% free Spike and Nucleocapsid mRNA in Test Formulation. Overall, this suggests high encapsulation of Spike and Nucleocapsid mRNA.

TABLE 5
mRNA encapsulation efficiency for Test Formulation

| Formulation | | Percent |
|---|---|---|
| sample | Injection | encapsulation (%) |
| 1 | Day 1 | 91.24 |
| 2 | Day 21 | 74.48 |

### Testing of Test Formulation.

Formulation was tested on the ZetaView for particle concentration, diameter, and poly-dispersity index (PDI). All parameters were as expected and are reported in Table 5 below.

TABLE 6

| | Concentration | Diameter | |
|---|---|---|---|
| Formulation | (particles/mL) | (nm) | PDI |
| S + N Test Formulation day 1 | 1.19E+10 | 127 | 0.16 |
| S + N Test Formulation day 21 | 2.50E+10 | 158 | 0.15 |

### Bioanalyzer RNA 6000 Nanochip.

Filtered gel and dye concentrate were equilibrated to RT for 30 minutes. 1 μL of RNA 6000 Nano dye concentrate was mixed with 65 μL of filtered gel. The solution was vortexed and centrifuged for 10 minutes at 13000×g. An RNA Nanochip was placed onto the chip priming station and 9 μL filtered gel-dye mixture was added to the well-marked G. The chip priming station was closed, and the plunger was pressed down. 9 μL of filtered gel-dye mixture was added to the two remaining wells marked G. 5 μL of RNA 6000 Nano marker was added to remaining the wells. 1 μL of RNA ladder and samples were added to the wells. The chip was vortexed at 2000 rpm for 1 minute. The chip was analyzed using the total RNA Nano Series II on the Agilent 2100 Bioanalyzer (FIG. 7). The electropherogram gives the size distribution profile of the RNA and it was used to generate the gel projection image. The gel project image was set to global scale. The concentration of the RNA was obtained from the output data.

mRNA Encapsulation Assay.

The Quant-iT RiboGreen™ RNA reagent and kit was used to quantify RNA in solution. Concentrated Quant-iT RiboGreen™ RNA reagent was diluted 200-fold with 1×TE buffer to generate the RiboGreen working solution. 16S and 23S ribosomal RNA from E. coli were diluted to a concentration of 50, 25, 5, 1, and 0 ng/mL and was used for the RNA standard curve. The amount of experimental sample was determined by diluting the samples and then determining whether the fluorescence intensity values fitted along the standard curve. All samples and standards were diluted in 1×TE buffer. 100 μL of diluted samples and standards and 100 μL of RiboGreen working solution were added to microplate wells. The plate was covered with aluminum foil and incubated for 3 minutes at room temperature. Samples were excited at 485 nm and emission was measured at 535 nm using a BioTek Cytation 5 microplate reader. RNA concentration vs. fluorescence emission intensity was plotted and the concentration of free RNA in solution were determined by the standard curve. The percentage of free RNA in solution was used to calculate the percentage of Spike and Nucleocapsid mRNA that is incorporated into the exosomes. 100 mg/ml Spike and Nucleocapsid mRNA (50 mg of each mRNA) was used as the input mRNA. The percentage of free RNA in solution is inversely correlated with the percentage encapsulated in exosomes.

### Flanking Untranslated Regions.

To produce in vitro-synthesized mRNAs, open reading frames (ORFs) that encode proteins of interest (POIs) are inserted into the plasmid pT7CypApoV6, a small bacterial episome that carries the ampicillin-resistance gene and a bacterial origin of replication, as well as the following elements, which are together designed to facilitate in vitro mRNA synthesis of the desired mRNA and in vivo translation of the POI from the in vitro-synthesized mRNA. In order, These elements include, in order and with all nt sequences are in bold, typed 5′ to 3′:
the T7 promoter (TAATACGACTCACTATAAG);
a GA dinucleotide, for in vitro synthesis using certain 5-nucleotide cap analogs;
the tetranucleotide TAAT, to enhance T7-mediated mRNA synthesis (1);
the 33 nucleotide-long 5′ untranslated region (UTR) from the CYP2E1 gene (CTCCCGGGCTG GCAGCAGGGCCCCAGCGGCACC) (2);
the sequence GGTACCGCGATCGCCGTATCATAAAGGCCGGTTACGGTAC GCGTTAATAGTGACTCTCGAGGGATCC, which contains KpnI and AsiSI sites (underlined) upstream of a stuffer fragment (italicized) and followed by an MluI site (underlined), two stop codons, and XhoI, and BamHI sites (underlined);
the 3′ UTR from the ApoA2 gene (AGTGTCCAGACCATTGTCTTCCAACCC CAGCTGGCCTCcAGAACACCCACTGGCCAGTCCTAGAGCTCCTGTCCCTACCCAC TCTTTGCTACAATAAATGCTGAATGAATCCA) (2);
the sequence AGATCTAAACCCTTGCGGCCGCAACGCGCGCG, containing BglII, NotI and MluI sites, all underlined; and
the T7 terminator sequence CTAGCATAACCCCTTGGGGCCTCTAAACGGGTC TTGAGGGGTTTT TTG.

This arrangement of sequences promotes efficient in vitro mRNA synthesis using the T7 polymerase and associated capping and polyadenylation enzymes (1), as well as the efficient translation of the mRNA into protein in human (or other) cells, due to the translation-enhancing properties of the 5′ and 3′ UTR sequences (2).

Although the invention has been described with reference to the above examples, it will be understood that modifications and variations are encompassed within the spirit and scope of the invention. Accordingly, the invention is limited only by the following claims.

### REFERENCES

ADDIN EN.REFLIST 1. Conrad, T., Plumbom, I., Alcobendas, M., Vidal, R., and Sauer, S. (2020) Maximizing transcription of nucleic acids with efficient T7 promoters. Commun Biol 3, 439
2. Asrani, K. H., Farelli, J. D., Stahley, M. R., Miller, R. L., Cheng, C. J., Subramanian, R. R., and Brown, J. M. (2018) Optimization of mRNA untranslated regions for improved expression of therapeutic mRNA. RNA Biol 15, 756-762

1. An extracellular vesicle (“EV”)-based nucleic acid composition, comprising one or more EVs each loaded with one or more polynucleotides each encoding the Wuhan-1 strain SARS-CoV-2 spike protein (SEQ ID NO:1) (“S protein”) or a variant of the S protein (“S protein variant”), and one or more polynucleotides each encoding the Wuhan-1 strain SARS-CoV-2 nucleocapsid protein (SEQ ID NO: 12) (“N protein”) or a variant of the N protein (“N protein variant”).

2. The EV-based nucleic acid composition of claim 1, wherein said polynucleotides are designed, or configured, both to be expressed, and to induce a humoral immune response and/or a cellular immune response, in an animal subject.

3. The EV-based nucleic acid composition of claim 1 or claim 2, wherein the S protein has the amino acid sequence according to SEQ ID NO:1.

4. The EV-based nucleic acid composition of claim 1 or claim 2, wherein the S protein variant comprises a di-proline substitution of 986KV987-to-986PP987 (“S-2P”).

5. The EV-based nucleic acid composition of any of claims 1, 2 and 4, wherein the S protein variant comprises a modification in its C-terminus that improves its cell-surface expression as compared to the S protein.

6. The EV-based nucleic acid composition of claim 4, wherein the S protein variant comprising the S-2P mutation has the amino acid sequence according to SEQ ID NO:2, or has at least 90%, 91%, 92%, 93%, 94%, 95%, 96%, 97% 98%, or 99% sequence identity with SEQ ID NO:2 while retaining the S-2P mutation.

7. (canceled)

8. The EV-based nucleic acid composition of claim 5, wherein said modification in the C-terminus of the S protein variant that improves its cell-surface expression comprises removal of one or more retrieval signals that serve to localize the S protein to a specific cellular compartment and/or expression of the extracellular domain of S protein as a fusion protein to a C-terminally positioned glycosylphosphatidylinositol (“GPI”) anchor-conferring peptide.

9. (canceled)

10. The EV-based nucleic acid composition of claim 1, further comprising a C-terminally positioned glycosvlphosphatidylinositol (GPI) anchor conferring peptide comprising a sequence PNKGSGTTSGTTRLLSGHTCFTLTGLLGTLVTMG LLTCOOH (SEQ ID NO:16).

11. The EV-based nucleic acid composition of claim 5, wherein said modification in the C-terminus of the S protein variant that improves its cell-surface expression comprises the sequence Tyr19-Thr20-Asp21-Ile22-Glu23-Met24 (SEQ ID NO:17) of the vesicular stomatitis virus glycoprotein (“VSV-G”) tail peptide, the VSV-G tail peptide sequence KLKHTKKRQIYTDIEMNRLGKCOOH (SEQ ID NO:18) (“VT”), or the tyrosine-to-alanine substituted form of the VT (“VTYA” [KLKHTKKRQIATDIEMNRLGKCOOH (SEQ ID NO:19)]).

12. The EV-based nucleic acid composition of any of claim 1, wherein a sequence KFDEDDSEPVLKGVKLHYTCOOH (SEQ ID NO:20) in the C-terminus of the S protein is removed.

13. The EV-based nucleic acid composition of claim 1, wherein the S protein variant comprises an amino acid change of D614G (“SD614G”).

14. The EV-based nucleic acid composition of claim 1, wherein the S protein variant comprises a cleavage site mutation of 682RRAR685-to-682GSAG685 (“S-CSM”).

15. The EV-based nucleic acid composition of claim 1, wherein the S protein variant is S-2P-VTYA having the amino acid sequence according to SEQ ID NO:3, or having at least 90%, 91%, 92%, 93%, 94%, 95%, 96%, 97% 98%, or 99% sequence identity with SEQ ID NO:3 while retaining the S-2P and VTYA mutations.

16. The EV-based nucleic acid composition of claim 1, wherein the S protein variant is SD614G-2P-VTYA having the amino acid sequence according to SEQ ID NO:4, or having at least 90%, 91%, 92%, 93%, 94%, 95%, 96%, 97% 98%, or 99% sequence identity with SEQ ID NO:4 while retaining the SD614G, S-2P and VTYA mutations.

17. The EV-based nucleic acid composition of claim 1, wherein the S protein variant is the Delta variant spike protein (“SDelta”) having the amino acid sequence according to SEQ ID NO:5, or having at least 90%, 91%, 92%, 93%, 94%, 95%, 96%, 97% 98%, or 99% amino acid sequence identity with SEQ ID NO:5 while retaining the SDelta mutation.

18. The EV-based nucleic acid composition of claim 1, wherein the S protein variant is SDelta-2P-VTYA having the amino acid sequence according to SEQ ID NO:6, or having at least 90%, 91%, 92%, 93%, 94%, 95%, 96%, 97% 98%, or 99% sequence identity with SEQ ID NO:6 while retaining the SDelta, S-2P and VTYA mutations.

19. The EV-based nucleic acid composition of claim 1, wherein the S protein variant is SD614G(ECD)-GPI having the amino acid sequence according to SEQ ID NO:7, or having at least 90%, 91%, 92%, 93%, 94%, 95%, 96%, 97% 98%, or 99% sequence identity with SEQ ID NO:7 while retaining the SD614G and GPI anchor mutations.

20. The EV-based nucleic acid composition of claim 1, wherein the S protein variant is SD614G-VT having the amino acid sequence according to SEQ ID NO:8, or having at least 90%, 91%, 92%, 93%, 94%, 95%, 96%, 97% 98%, or 99% sequence identity with SEQ ID NO:8 while retaining the SD614G and VT mutations.

21. The EV-based nucleic acid composition of claim 1, wherein the S protein variant is SD614G-VTYA having the amino acid sequence according to SEQ ID NO:9, or having at least 90%, 91%, 92%, 93%, 94%, 95%, 96%, 97% 98%, or 99% sequence identity with SEQ ID NO:9 while retaining the SD614G and VTYA mutations.

22. The EV-based nucleic acid composition of claim 1, wherein the S protein variant is SD614G-2P having the amino acid sequence according to SEQ ID NO:10, or having at least 90%, 91%, 92%, 93%, 94%, 95%, 96%, 97% 98%, or 99% sequence identity with SEQ ID NO:10 while retaining the SD614G and S-2P mutations.

23. The EV-based nucleic acid composition of claim 1, wherein the S protein variant is SD614G-2P(ECD)-GPI having the amino acid sequence according to SEQ ID NO:11, or having at least 90%, 91%, 92%, 93%, 94%, 95%, 96%, 97% 98%, or 99% sequence identity with SEQ ID NO:11 while retaining the SD614G, S-2P and GPI anchor mutations.

24. The EV-based nucleic acid composition of any of claim 1, wherein the N protein has the amino acid sequence according to SEQ ID NO:12, or has at least 90%, 91%, 92%, 93%, 94%, 95%, 96%, 97% 98%, or 99% amino acid sequence identity with SEQ ID NO:12.

25. The EV-based nucleic acid composition of claim 1, wherein the N protein variant is Lamp-N-Lamp protein (“LNL protein”) having the amino acid sequence according to SEQ ID NO:13.

26. The EV-based nucleic acid composition of claim 1, wherein said one or more polynucleotides are each a messenger RNA (“mRNA”).

27-29. (canceled)

30. The EV-based nucleic acid composition of claim 1, wherein said EV is derived from 293F cells, cardiospheres, cardiosphere-derived cells (CDCs), activated-specialized tissue-effector cells (ASTECs), or mesenchymal stem cells (MSCs).

31. The EV-based nucleic acid composition of claim 1, wherein said polynucleotide contains a codon-optimized open reading frame (“ORF”).

32. The EV-based nucleic acid composition of claim 1, wherein said polynucleotide contains an optimized three prime untranslated region (“3′UTR”) and/or an optimized five prime untranslated region (“5′UTR”).

33. The EV-based nucleic acid composition of claim 1, wherein said polynucleotide contains a polyadenylated tail.

34. The EV-based nucleic acid composition of claim 1, wherein said EVs are exosomes or microvesicles.

35. A method of making the EV-based nucleic acid composition of claim 1, the method comprising contacting one or more EVs with the one or more polynucleotides each encoding the S protein or the S protein variant according to claim 1, and the one or more polynucleotides each encoding the N protein or the N protein variant according to claim 1, in the presence of a chemical lipofection reagent, whereby said polynucleotides are loaded into said EVs.

36. The method of claim 35, wherein said chemical lipofection reagent is a polycationic lipid.

37-38. (canceled)

39. A method of inducing an immune response, or immunizing, against the S protein or the S protein variant in a subject, comprising administering the EV-based nucleic acid composition according to claim 1 to the subject in an effective amount to produce the immune response.

40-54. (canceled)
